SEQUENCE LISTING (6 sequences)

SEQ ID NO: 1 - protein, 112 aa, synthetic construct
QIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSV

SEQ ID NO: 2 - protein, 200 aa, synthetic construct
QIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIP

SEQ ID NO: 3 - protein, 629 aa, synthetic construct
QIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPE

SEQ ID NO: 4 - protein, 447 aa, synthetic construct
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 427 aa, synthetic construct
QIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1344 bp, synthetic construct
  source 1..1344: mol_type = other DNA
atggacaagttctggtggcacgcagcatggggactgtgcctggtgccactgagcctggcccagatcgatctgaacatcacctgtaggttcgccggcgtgtttcacgtggagaagaatggcaggtactccatctctcgcacagaggcagccgacctgtgcaaggcctttaactctaccctgcccacaatggcccagatggagaaggccctgagcatcggcttcgagacctgccggtacggctttatcgagggccacgtggtcatcccaagaatccaccccaattccatctgtgccgccaacaataccggcgtgtatatcctgacctctaacacaagccagtacgatacatattgcttcaatgcctctgccccccctgaggaggattgtaccagcgtgacagacctgcctaacgcctttgatggcccaatcaccatcacaatcgtgaatcgggacggcaccagatacgtgcagaagggcgagtataggacaaaccctgaggacatctaccctagcaatccaaccgacgatgacgtgagcagcggctctagctccgagcggagcagcaccagcggaggatacatcttctataccttttctacagtgcaccctatcccagatgaggacagcccctggatcaccgattccacagacagaatccccgacaagacccacacatgcccaccatgtccagcacctgagctgctgggaggaccatccgtgttcctgtttcctccaaagcctaaggataccctgatgatctccagaacccccgaggtgacatgcgtggtggtggacgtgagccacgaggaccctgaggtgaagttcaactggtacgtggacggcgtggaggtgcacaatgccaagaccaagccccgggaggagcagtacaactctacctatagagtggtgagcgtgctgacagtgctgcaccaggattggctgaacggcaaggagtataagtgcaaggtgagcaataaggccctgccagcccccatcgagaagaccatctccaaggcaaagggacagcccagggagcctcaggtgtacacactgccccctagccgcgaggagatgaccaagaaccaggtgtccctgacatgtctggtgaagggcttctatcctagcgacatcgccgtggagtgggagtccaatggccagccagagaacaattacaagaccacaccacccgtgctggattccgacggctctttctttctgtattccaagctgaccgtggataagtctcggtggcagcagggcaacgtgtttagctgttccgtgatgcacgaggccctgcacaatcactacacacagaagtctctgagcctgtcccccggcaagtaa

(19) United States
(12) Patent Application Publication
(10) Pub. No.: US 2024/0277792 A1
(43) Pub. Date: Aug. 22, 2024

(54) ONCOLYTIC VIRUSES AND METHODS OF USE THEREOF FOR THE TREATMENT OF CANCER

(71) Applicant: The Board of Regents of The University of Texas System, Austin, TX (US)

(72) Inventors: Balveen Kaur, Augusta, GA (US); Bangxing Hong, Augusta, GA (US); Upasana Sahu, Augusta, GA (US)

(21) Appl. No.: 18/653,847

(22) Filed: May 2, 2024

Related U.S. Application Data
(60) Provisional application No. 62/499,521, filed on Jan. 30, 2017.

(51) Int. Cl. A61K 35/763; A61K 45/06; A61P 35/00; C07K 14/705; C12N 15/86

(52) U.S. Cl. CPC A61K 35/763; A61K 45/06; A61P 35/00; C07K 14/70585; C12N 15/86; C12N 2710/16622; C12N 2710/16632; C12N 2710/16643; C12N 2710/16671

(57) ABSTRACT

The present disclosure relates to an oncolytic virus and methods of using such to treat cancer. The methods may advantageously involve further treating with at least a first DNA damaging agent, such as radiation or a chemotherapeutic agent. In some embodiments, the oncolytic virus is an oncolytic herpes simplex virus (oHSV) that optionally has been engineered to expression a portion of CD44, in particular the extracellular domain of CD44.

### CROSS-REFERENCE TO RELATED APPLICATION

The present application claims priority to U.S. Application No. 62/499,521, filed May 2, 2023, the disclosure of which is specifically incorporated by reference herein in its entirety.

### REFERENCE TO THE SEQUENCE LISTING

The Sequence Listing XML submitted as a file named “2022-0029_ST26.xml” and having a size of 9,298 bytes is hereby incorporated by reference pursuant to 37 C.F.R. § 1.834(c)(1).

### FIELD OF THE INVENTION

The present disclosure relates generally to the field of biology and medicine. More particularly, it concerns methods for the treatment of cancer, including, but not limited to, brain cancer.

### BACKGROUND OF THE INVENTION

Gliomas arise from glial cells within the central nervous system and the World Health Organization has classified them to guide prognosis and treatment paradigms (Wesseling, P. and D. Capper, WHO 2016 Classification of gliomas. Neuropathol Appl Neurobiol, 2018. 44(2): p. 139-150). Glioblastoma (WHO Grade IV) is a challenging subset of gliomas given their aggressive nature, resistance to chemotherapy/radiation from tumor heterogeneity, and difficulty penetrating the blood-brain barrier (Noch, E. K., R. Ramakrishna, and R. Magge, Challenges in the Treatment of Glioblastoma: Multisystem Mechanisms of Therapeutic Resistance. World Neurosurg, 2018. 116: p. 505-517). Even with aggressive treatment with surgical resection, chemotherapy, radiation mean survival is less than 2 years (Thakkar, J. P., et al., Epidemiologic and molecular prognostic review of glioblastoma. Cancer Epidemiol Biomarkers Prev, 2014. 23(10): p. 1985-96). Oncolytic viruses (OV) have been evaluated for use in treatment of many solid tumors as they are able to replicate within tumor cells, induce tumor cell death, and also activate anti-tumor host immune response (Martikainen, M. and M. Essand, Virus-Based Immunotherapy of Glioblastoma. Cancers (Basel), 2019. 11(2)). Oncolytic HSV is a promising biotherapy being explored as a treatment for GBM. In recent years, the FDA has approved the use of Talimogene Laherparepvec for treatment of melanoma which is a promising prospect for other oncolytic viral therapies. Among the limitations of OV therapy is included a limited understanding of the tumor extracellular matrix (ECM) changes upon viral infection and the impact of these changes on therapy.

Thus, there remains a need for improvements in the field of oncolytic viral therapy.

Therefore, it is an object of the invention to provide an improved understanding the mechanisms underlying OV therapy and improved OVs engineered based thereon.

### SUMMARY OF THE INVENTION

Oncolytic viruses engineered to express a soluble CD44, and methods of thereof use in the treatment of cancer are provided.

In some embodiments, the oncolytic virus is an oncolytic herpes simplex virus (oHSV). In some aspects, the oHSV is an HSV-1 strain. In some aspects, the oHSV contains a nucleic acid sequence encoding at least a portion of CD44. In further aspects, the oHSV expresses the extracellular domain of CD44. In additional aspects, the oHSV secretes soluble CD44.

In some embodiments, the present disclosure provides methods of treating a subject having a cancer including administering an effective amount of an oncolytic virus to the subject alone or in conjunction with at least a first DNA damaging agent. In some aspects, the DNA damaging agent is a DNA-damaging agent is radiation. In some aspects said DNA damaging agent is a chemotherapeutic agent.

In additional aspects, the subject is administered a CD44-expressing oHSV in combination with radiation therapy. In additional aspects, the subject is administered a CD44-expressing oHSV in combination with an immune checkpoint inhibitor.

In some aspects, the amount of the oncolytic virus is effective at stimulating immune cell infiltration of the tumor. In some aspects, the amount of the oncolytic virus is effective at reducing the number of stem cells present in a tumor. In some aspects, the amount of the oncolytic virus is effective for inducing stem cell differentiation. In some aspects, the amount of the oncolytic virus is effective at increasing the replication and infectivity of said oncolytic virus. In some aspects, the amount of the oncolytic virus is effective at inducing DNA damage in an infected tumor cell. In some aspects, the amount of the oncolytic virus is effective at downregulating MAPK signaling in an infected tumor cell. In some aspects, the amount of the oncolytic virus is effective at inducing DNA damage in an infected tumor cell.

In some embodiments, are methods of treating radiation-resistant brain cancer in a subject including administering an effective amount of an oncolytic herpes simplex virus (oHSV) and a radiation therapy to the subject. In other embodiments, are pharmaceutical compositions including an oncolytic herpes simplex virus expresses the extracellular domain of CD44.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A is an illustration of the results of RNA sequencing of glioma cells revealing an enrichment of pathways related to tumor and extracellular matrix interactions. FIGS. 1B-1C are Kaplan-Meier plots of gene expression of Hyaluronan Synthase (HAS, enzyme that makes hyaluronan, FIG. 1B) and Hyaluronidase (HYAL, key HA degrading enzyme, FIG. 1C) showing that patients with higher HAS had a worse prognosis and patients with a higher amount of HYAL had a better prognosis. FIG. 1D is a Kaplan-Meier survival curve from TCGA-dataset of grade IV primary tumors around 400 patients further showed that higher CD44 expression correlated with a worse prognosis for GBM patients. FIG. 1E is a dot plot showing CD44 gene expression in tumor tissue derived from GBM patients compared to normal brain. FIG. 1F is a dot plot showing CD44 mRNA expression levels among GBM transcriptional subtypes. FIG. 1G is a plot showing the correlation between CD44 expression and stromal scores of patients.

FIG. 1H is a series of scatter plots showing CD44 expression co-staining with stem cell marker CD133 in GBM neurosphere cultures.

FIG. 2A is an illustration of the genetic structure of oHSV-sCD44: an oHSV encoding for sCD44 backbone. FIG. 2B is a bar graph showing the results of ELISA probing for sCD44 in conditioned medium of glioma cells infected with oHSV-sCD44. FIGS. 2C-2D are plots showing quantifying the spread of GFP +ve infected cells over time by live cell fluorescent imaging using Cytation 5 Cell Imaging Multi-Mode Reader connected to a BioSpa 8 Automated Incubator (BioTek Instruments, INC.). FIGS. 2E and 2F are each a series of flow cytometric scatter plots for GFP⁺ cells and live dead staining for infection (FIG. 2E) and tumor cell lysis (FIG. 2F) by oHSV-sCD44 as compared to oHSVctl in both GBM12 and GBM28 cells.

FIG. 3A is a pair of bar graphs illustrating the results of a colony formation assay performed to detect the ability of oHSV-sCD44 or oHSVctl infected cells to grow and form colonies in an anchorage independent manner. FIG. 3B is a bar graph showing the transcript levels of Sox2 and MMP2 in mock, oHSVctl, and oHSV-sCD44 infected cells. FIG. 3C is a pair of bar graphs showing expression of stem cell marker Sox2 (right) and differentiation marker GFAP (left). FIG. 3D is a series of scatter plots showing the results of flow cytometry analysis of infection of CD133-ve cells and CD133+ve cells oHSV-sCD44 and oHSVctl. FIG. 3E is a series of micrographs illustrating, and a corresponding graph quantitating, the results of an in vitro limited dilution assay investigating the impact of oHSV secreted sCD44 on GMB tumor initiating stem cell population.

FIGS. 4A and 4B are survival curves of tumor bearing mice: murine 005 model (FIG. 4A) and DB7 breast cancer brain metastases model (FIG. 4B) treated with a single dose of control oHSV-ctl or oHSV-sCD44. FIG. 4C is a bar graph showing the results of HIF Reporter analysis using a HIF responsive promotor driven luciferase also showed that oHSV-sCD44 reduced HIF activity for mock, oHSV-ctl, or oHSV-sCD44. FIG. 4D is an image of a Western blot analysis showing CD44-ICD generation in primary GBM cells infected with oHSV-sCD44 as compared to oHSV-Ctl infected cells. FIG. 4E is an image of a Western blot analysis of HIF 1/2 stabilization and MAPK activation in GBM cell lysates treated with oHSV-Ctl or oHSV-sCD44. FIG. 4F is an image of Western blot analysis of HIF1α, SOX2 and pERK1/2 following treatment with HIF1α inhibitor in combination with oHSV-sCD44.

FIG. 5A is a bar graph showing the results of flow cytometry analysis of total cells number of T cells infiltration following oHSV-sCD44 intra-tumoral injection. FIGS. 5B and 5C are scatter plots showing the results of flow cytometry analysis of Immune profiles (e.g., CD45) (FIG. 5B) and CD11b+CD45+ cells with concurrent increase in CD45+CD11b− lymphocytes significantly (FIG. 5C) in 005 model.

FIGS. 6A and 6B are plots showing total RNA sequencing for DNA repair pathways (FIG. 6A) and cellular response to radiation (FIG. 6B) of GBM 28 glioma cells infected with oHSV-ctl or oHSV-sCD44. FIG. 6C is an image of the results of a Western blot showing phosphorylation of H2Ax and DNA-PKC in cells treated with oHSV-sCD44 relative to HSV Ctrl-treated cells.

FIG. 7A is an image showing the results of a Western blot probing the DNA damage and impaired DNA damage repair (phosphorylation of H2AX (γH2AX)) following a combination of oHSV-sCD44 infection and radiation. FIG. 7B is a bar graph and FIG. 7C is an image of the results of a Western blot showing the stemness markers Sox2 and Nestin following a combination of oHSV-sCD44 infection and radiation. FIG. 7D is an experimental scheme showing mice with human GBM12 or murine 005 glioma cells implanted intracranially were treated with oHSV-Ctr or oHSV-sCD44 with irradiation and survival of mice was monitored. FIGS. 7E and 7F are Kaplan-Meier curves of survival of these mice.

FIG. 8 is an experimental scheme for analysis of anti-tumor efficacy of oHSV-sCD44 in combination with irradiation.

### DETAILED DESCRIPTION OF THE INVENTION

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating certain embodiments of the present disclosure, are given by way of illustration only, since various changes and modifications within the spirit and scope of the present disclosure will become apparent to those skilled in the art from this detailed description.

### II. Compositions

Gliomas are primary brain tumors of the central nervous system and the World Health Organization has classified them to guide prognosis and treatment paradigms. WHO Grade IV glioma are a challenging subset of gliomas given their aggressive nature, resistance to radiation/chemotherapy from tumor heterogeneity, and difficulty penetrating the blood-brain barrier. Even with aggressive treatment with surgical resection, chemotherapy and radiation patient mean survival is less than 2 years. Oncolytic viruses (OV) have been evaluated for use in treatment of many solid tumors as they are able to replicate within tumor cells, induce tumor cell death, and also activate anti-tumor host immune response (Martikainen, M. and M. Essand, Virus-Based Immunotherapy of Glioblastoma. Cancers (Basel), 2019. 11(2)). The limitations of OV therapy include a limited understanding of the tumor ECM changes upon viral infection and the impact of these changes on therapy. Provided herein is evidence of a significant increase in hyaluronan production in brain tumors following virotherapy.

Hyaluronic acid (HA) the major glycosaminoglycan in the extracellular matrix (ECM) has long been associated with tumor progression, for example, prostate cancer and glioblastoma (GBM).

The experiments below reveal a significant increase in hyaluronan production in brain tumors after virotherapy. The major receptor that transduces hyaluronan signaling is CD44. Provided herein are OV expressing CD44 and methods of use thereof as an anti-cancer therapy.

CD44 is widely expressed in many cancer types and has high expression in Glioblastoma. These properties make CD44 a suitable target for anti-cancer therapy. The experiments below illustrate the discovery that increased secretion of HA after HSV therapy increases the tumor cell dependence on CD44 signaling, and its blockade hence sensitizes tumor cells to virus therapy with and without radiation. Results show that CD44 is expressed in both GBM and GSC in vitro and in vivo, and establish a correlation of CD44 with GBM prognosis. Specifically TCGA analysis showed that high levels of CD44 in GBM patients correlates with a worse prognosis. Thus, CD44's interaction with hyaluronan plays a significant role in GBM invasion and progression, and is a biomarker with prognostic value.

Thus, provided are OV, e.g., oncolytic HSV, that have been engineered to express and secrete soluble CD44 intended to function as a competitive inhibitor of CD44-HA interactions. In the experiments below, oHSV-sCD44 was demonstrated to induce the secretion of the extracellular domain of CD44 resulting in increased tumor lysis when tumor cells were infected with oHSV-sCD44.

Treatment of glioblastoma (GBM) stem cells (GSCs) with oHSV-sCD44 demonstrated that infection with oHSV-sCD44 disables the stemness of glioblastoma (GBM) stem cells (GSCs), reducing the GSC population in the GBM nanospheres. Infection with oHSV-sCD44 was also shown to significantly increase oncolytic virus infection and replication in both GSCs and GBM, to induce DNA damage and to modulate downregulates MAPK signaling which inhibits tumor growth. Thus, infection with oHSV-sCD44 increased the antitumor activity over infection with oHSV alone.

Oncolytic viral therapy with oHSV has been shown to be diminished by the host's immune response. It was also determined that infection with oHSV-sCD44 results in an increase in immune cell infiltration of the tumor.

In the experiments below, intra-tumoral injection of oHSV-sCD44 in a patient-derived primary GBM xenograft mouse model identified significant inhibition of tumor growth with reduced Sox2, CD44 and HA expression, and increased oHSV replication and tumor cell lysis in TME. Moreover, blocking HA-CD44 signaling with a single dose of oHSV-sCD44 in murine glioma syngeneic model induced a strong anti-tumor immune response with enhanced T cell infiltration. These findings indicate that HA-CD44 signaling is critical to maintaining the stem cell state of GSCs and that blocking it by oHSV-sCD44 triggers GSC differentiation, sensitizes GSCs to radiotherapy. The combination of oHSV-sCD44 with low dose radiation resulted in increased immune infiltration and enhanced survival in vivo. Indicating that oHSV-sCD44 is an oncolytic and immune stimulating anticancer therapeutic

Thus, described herein are therapeutic oncolytic viruses engineered to block HA-CD44 signaling in the tumor microenvironment (TME). Typically, the oncolytic virus, e.g., an oncolytic HSV, secretes an extracellular domain of CD44 (oHSV-sCD44). Preferably, it significantly reduces the stemness of glioblastoma stem cells (GSCs) by suppressing Sox2 and upregulating GFAP expression, and sensitizes the GSCs to radiation therapy.

Furthermore, infection with oHSV-sCD44 was found to sensitize cancer cells to radiotherapy, thus, providing evidence of enhanced antitumor activity using oHSV-sCD44, alone or in combination with radiation therapy. In some embodiments an oncolytic virus, such as an oncolytic Herpes Simplex virus, engineered to express a soluble extracellular portion of CD44 and in combination with radiotherapy synergistically increase tumor cell killing.

### A. Oncolytic Viruses

Oncolytic virus, e.g., HSV engineered to express a soluble CD44 are provided.

### 1. Soluble CD44

The soluble CD44 is believed to be excreted into the extracellular environment into the tumor microenvironment (TME) and function as a decoy receptor and prevents CD44 signaling leading to decreased tumor cell proliferation, migration, and adhesion.

CD44 is a transmembrane glycoprotein shown to have extra- and intra-cellular domains which are involved in cellular interactions with extracellular matrix (ECM). The CD44 glycoprotein serves as a mediator between the extracellular environment and downstream gene expression which determines downstream effects (Martin, T. A., et al., The role of the CD44/ezrin complex in cancer metastasis. Crit Rev Oncol Hematol, 2003. 46(2): p. 165-86). The extracellular domain is involved in adhesion, migration and inflammation and further interacts with Matrix Metallopeptidases (MMPs) which influences adhesion and migration of cells. CD44's interaction with hyaluronan (a glycosaminoglycan of the ECM) has been shown to be involved in cell adhesion and cell migration in tumor metastases and leukocyte migration during inflammation (Ponta, H., L. Sherman, and P.A. Herrlich, CD44: from adhesion molecules to signalling regulators. Nat Rev Mol Cell Biol, 2003. 4(1): p. 33-45).

A consensus sequence of human CD44 is known in the art, see, e.g., UniProt Reference number P16070·CD44_HUMAN, which is specifically incorporated by reference herein in its entirety, and provides that a CD44 extracellular domain is amino acids 21-649 of the full length protein:

(SEQ ID NO: 3)
QIDLNITCRFAGVFHVEKNGRYSISRTEAADLCKAFNSTLPTMAQME
KALSIGFETCRYGFIEGHVVIPRIHPNSICAANNTGVYILTSNTSQY
DTYCFNASAPPEEDCTSVTDLPNAFDGPITITIVNRDGTRYVQKGEY
RTNPEDIYPSNPTDDDVSSGSSSERSSTSGGYIFYTFSTVHPIPDED
SPWITDSTDRIPATTLMSTSATATETATKRQETWDWFSWLFLPSESK
NHLHTTTQMAGTSSNTISAGWEPNEENEDERDRHLSFSGSGIDDDED
FISSTISTTPRAFDHTKQNQDWTQWNPSHSNPEVLLQTTTRMTDVDR
NGTTAYEGNWNPEAHPPLIHHEHHEEEETPHSTSTIQATPSSTTEET
ATQKEQWFGNRWHEGYRQTPKEDSHSTTGTAAASAHTSHPMQGRTTP
SPEDSSWTDFFNPISHPMGRGHQAGRRMDMDSSHSITLQPTANPNTG
LVEDLDRTGPLSMTTQQSNSQSFSTSHEGLEEDKDHPTTSTLTSSNR
NDVTGGRRDPNHSEGSTTLLEGYTSHYPHTKESRTFIPVTSAKTGSF
GVTAVTVGDSNSNVNRSLSGDQDTFHPSGGSHTTHGSESDGHSHGSQ
EGGANTTSGPIRTPQIPE.

An exemplary soluble portion of the extracellular domain of CD44 is amino acids 1-200 of SEQ ID NO:3

(SEQ ID NO: 2)
QIDLNITCRFAGVFHVEKNGRYSISRTEAADLCKAFNSTLPTMAQME
KALSIGFETCRYGFIEGHVVIPRIHPNSICAANNTGVYILTSNTSQY
DTYCFNASAPPEEDCTSVTDLPNAFDGPITITIVNRDGTRYVQKGEY
RTNPEDIYPSNPTDDDVSSGSSSERSSTSGGYIFYTFSTVHPIPDED
SPWITDSTDRIP

It is believed that the following sequence is important for HA binding function of hCD44 (amino acids 1-112 of SEQ ID NO:3, (Päll, et al., Soluble CD44 interacts with intermediate filament protein vimentin on endothelial cell surface. PLOS One., 6(12):e29305, PMID: 22216242 (2011)):

(SEQ ID NO: 1)
QIDLNITCRFAGVFHVEKNGRYSISRTEAADLCKAFNSTLPTMAQME
KALSIGFETCRYGFIEGHVVIPRIHPNSICAANNTGVYILTSNTSQY
DTYCFNASAPPEEDCTSV.

In some embodiments, the sCD44 of the virus is a fusion protein including at least a portion of the CD44 extracellular domain fused to one or more additional polypeptides, e.g., a heterologous polypeptide(s).

In some embodiments, an additional polypeptide is an Fc domain. The Fc domain may increase stability and half-life of the secreted protein.

In some embodiments, the sCD44 encoded by the virus includes a signal peptide. In some embodiments, the signal peptide is cleaved in the final form a mature secreted protein. The signal peptide can be the endogenous CD44 signal peptide or substituted with an alternative signal peptide.

An exemplary amino acid sequence for an hsCD44-Fc fusion protein:

(SEQ ID NO: 4)
MDKFWWHAAWGLCLVPLSLAQIDLNITCRFAGVFHVEKNGRYSISRT
EAADLCKAFNSTLPTMAQMEKALSIGFETCRYGFIEGHVVIPRIHPN
SICAANNTGVYILTSNTSQYDTYCFNASAPPEEDCTSVTDLPNAFDG
PITITIVNRDGTRYVQKGEYRTNPEDIYPSNPTDDDVSSGSSSERSS
TSGGYIFYTFSTVHPIPDEDSPWITDSTDRIPDKTHTCPPCPAPELL
GGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALP
APIEKTISKAKGQPREPQVYTLPPSREEMTKNQVSLTCLVKGFYPSD
IAVEWESNGQPENNYKTTPPVLDSDGSFFLYSKLTVDKSRWQQGNVF
SCSVMHEALHNHYTQKSLSLSPGK.

A nucleic acid sequence encoding SEQ ID NO:4 is

(SEQ ID NO: 6)
ATGGACAAGTTCTGGTGGCACGCAGCATGGGGACTGTGCCTGGTGCC
ACTGAGCCTGGCCCAGATCGATCTGAACATCACCTGTAGGTTCGCCG
GCGTGTTTCACGTGGAGAAGAATGGCAGGTACTCCATCTCTCGCACA
GAGGCAGCCGACCTGTGCAAGGCCTTTAACTCTACCCTGCCCACAAT
GGCCCAGATGGAGAAGGCCCTGAGCATCGGCTTCGAGACCTGCCGGT
ACGGCTTTATCGAGGGCCACGTGGTCATCCCAAGAATCCACCCCAAT
TCCATCTGTGCCGCCAACAATACCGGCGTGTATATCCTGACCTCTAA
CACAAGCCAGTACGATACATATTGCTTCAATGCCTCTGCCCCCCCTG
AGGAGGATTGTACCAGCGTGACAGACCTGCCTAACGCCTTTGATGGC
CCAATCACCATCACAATCGTGAATCGGGACGGCACCAGATACGTGCA
GAAGGGCGAGTATAGGACAAACCCTGAGGACATCTACCCTAGCAATC
CAACCGACGATGACGTGAGCAGCGGCTCTAGCTCCGAGCGGAGCAGC
ACCAGCGGAGGATACATCTTCTATACCTTTTCTACAGTGCACCCTAT
CCCAGATGAGGACAGCCCCTGGATCACCGATTCCACAGACAGAATCC
CCGACAAGACCCACACATGCCCACCATGTCCAGCACCTGAGCTGCTG
GGAGGACCATCCGTGTTCCTGTTTCCTCCAAAGCCTAAGGATACCCT
GATGATCTCCAGAACCCCCGAGGTGACATGCGTGGTGGTGGACGTGA
GCCACGAGGACCCTGAGGTGAAGTTCAACTGGTACGTGGACGGCGTG
GAGGTGCACAATGCCAAGACCAAGCCCCGGGAGGAGCAGTACAACTC
TACCTATAGAGTGGTGAGCGTGCTGACAGTGCTGCACCAGGATTGGC
TGAACGGCAAGGAGTATAAGTGCAAGGTGAGCAATAAGGCCCTGCCA
GCCCCCATCGAGAAGACCATCTCCAAGGCAAAGGGACAGCCCAGGGA
GCCTCAGGTGTACACACTGCCCCCTAGCCGCGAGGAGATGACCAAGA
ACCAGGTGTCCCTGACATGTCTGGTGAAGGGCTTCTATCCTAGCGAC
ATCGCCGTGGAGTGGGAGTCCAATGGCCAGCCAGAGAACAATTACAA
GACCACACCACCCGTGCTGGATTCCGACGGCTCTTTCTTTCTGTATT
CCAAGCTGACCGTGGATAAGTCTCGGTGGCAGCAGGGCAACGTGTTT
AGCTGTTCCGTGATGCACGAGGCCCTGCACAATCACTACACACAGAA
GTCTCTGAGCCTGTCCCCCGGCAAGTAA

The sequence following the soluble domain in SEQ ID NO:4, i.e., amino acids 221-447, is the Fc domain.

The sequence preceding the soluble domain in SEQ ID NO:4, i.e., amino acids 1-20, is a signal peptide sequence, which can be substituted with an alternative signal peptide sequence. Thus, another exemplary fusion protein is

(SEQ ID NO: 5)
QIDLNITCRFAGVFHVEKNGRYSISRTEAADLCKAFNSTLPTMAQME
KALSIGFETCRYGFIEGHVVIPRIHPNSICAANNTGVYILTSNTSQY
DTYCFNASAPPEEDCTSVTDLPNAFDGPITITIVNRDGTRYVQKGEY
RTNPEDIYPSNPTDDDVSSGSSSERSSTSGGYIFYTFSTVHPIPDED
SPWITDSTDRIPDKTHTCPPCPAPELLGGPSVFLFPPKPKDTLMISR
TPEVTCVVVDVSHEDPEVKFNWYVDGVEVHNAKTKPREEQYNSTYRV
VSVLTVLHQDWLNGKEYKCKVSNKALPAPIEKTISKAKGQPREPQVY
TLPPSREEMTKNQVSLTCLVKGFYPSDIAVEWESNGQPENNYKTTPP
VLDSDGSFFLYSKLTVDKSRWQQGNVFSCSVMHEALHNHYTQKSLSL
SPGK,

optionally including a heterologous signal peptide sequence fused to the N-terminus thereof.

Thus, in some embodiments, the oncolytic virus encodes and expresses the amino acid sequence including any one of SEQ ID NOS: 1-5, or a functional fragment thereof, or a variant thereof having at least 70, 75, 80, 85, 90, or 95% sequence identity to any one of SEQ ID NOS: 1-5. “RY”, e.g., Arg-41, Tyr-42, Arg-78, Tyr-79 are important for interaction with HA (Bajorath, et al., Identification of CD44 residues important for hyaluronan binding and delineation of the binding site. J Biol Chem., 273(1):338-43, PMID: 9417085 (1988)). Thus, in some embodiments, one, two, three, or all four of these amino acid residues are present. In some embodiments, one, two, three, or all four of these residues are present and not mutated.

### 2. Oncolytic Virus Backbones

Provided herein are OV expressing soluble CD44. Various OV backbones can be engineered to express the soluble CD44. OVs are derived from single-or double-stranded DNA or RNA viruses according to nucleic acid type. See, e.g., Lin, D., Shen, Y. & Liang, T. “Oncolytic virotherapy: basic principles, recent advances and future directions.” Sig Transduct Target Ther 8, 156 (2023). doi.org/10.1038/s41392-023-01407-6, which is specifically incorporated by reference in its entirety. ssRNA and dsDNA viruses are the most prevalent in OVs products, except for reovirus (dsRNA) and parvovirus (ssDNA). dsDNA viruses mainly include adenovirus, vaccinia virus, herpesvirus, etc., while ssRNA viruses are composed of two main categories: positive-sense (coxsackievirus, Seneca Valley virus, poliovirus) and negative-sense (measles virus, Newcastle Disease virus, vesicular stomatitis virus). The genetic information of positive-sense ssRNA viruses is directly translated into protein by ribosomes of host cells, while the nucleic acid of negative-sense ssRNA viruses is complementary to the viral mRNA, which must be transcribed into positive-sense RNA before it can be translated into protein. OVs can also be divided into naturally attenuated viral strains and genetically modified viral vectors according to their structures.

Thus, the sCD44 can be engineered into an adenovirus, vaccinia virus, herpesvirus, coxsackievirus, Seneca Valley virus, poliovirus, measles virus, Newcastle Disease virus, vesicular stomatitis virus, Herpes simplex virus (HSV) (e.g., HSV-1 and HSV-2). The virus's backbone (before engineering of the sCD44 transgene) can be one previously identified as oncolytic. It can be naturally occurring or previously engineered.

Herpes simplex virus (HSV), an enveloped virus with dsDNA protected by the nucleocapsid, and surrounded by the tegument, has two specific serotypes (Yuan, S. et al. Cryo-EM structure of a herpesvirus capsid at 3.1 Å. Science 360, eaao7283 (2018)). HSV contains a large genome of at least 150 kb and a complex structure, which provides the possibility for the insertion of relatively large fragments and multiple transgenes (Manservigi, et al. HSV recombinant vectors for gene therapy. Open Virol. J. 4, 123-156 (2010)). Four major viral glycoproteins, gB, gD, gH and gL, are expressed on the surface of the HSV envelope enabling the binding with various cellular receptors (Agelidis, et al. Cell entry mechanisms of HSV: what we have learned in recent years. Future Virol. 10, 1145-1154 (2015)). During infection, the envelope fuses with lipid bilayers of the cell membrane to expose the nucleocapsid to the nuclear membrane (Weed, D. J. & Nicola, A. V. Herpes simplex virus membrane fusion. Adv. Anat. Embryol. Cell Biol. 223, 29-47 (2017)). The viral genome is then released into the cytosol, and transported into the nucleus where transcription initiates. The viral gene transcription and protein synthesis are strictly regulated by the herpesvirus genome. According to the order of transcription and translation, viral proteins are divided into immediate-early proteins, early proteins and late proteins (Maruzuru, Y. et al. Role of herpes simplex virus 1 immediate early protein ICP22 in viral nuclear egress. J. Virol. 88, 7445-7454 (2014)), in which modifying the genes encoded by these proteins is a common method. As a cytolytic virus, HSV can infect multiple types of cancer cells and quickly replicate, spreading the progeny viruses easily within neoplasms (Shen, Y. & Nemunaitis, J. Herpes simplex virus 1 (HSV-1) for cancer treatment. Cancer Gene Ther. 13, 975-992 (2006)). In addition, anti-HSV drugs like Acyclovir can be utilized to ensure the safety of oncolytic HSV (oHSV) to counteract virulence (Hartkopf, et al. Oncolytic virotherapy of gynecologic malignancies. Gynecol. Oncol. 120, 302-310 (2011)). Even though more than half of the population possesses neutralizing antibodies against HSV, it can still evade the host immunity through different mechanisms, rendering it a model for an ideal OV vector (Mozzi, A. et al. Simplexviruses successfully adapt to their host by fine-tuning immune responses. Mol. Biol. Evol. 39, msac 142 (2022)). Currently, HSV-1 is one of the most commonly used strains of OVs. The representative works include T-VEC (Johnson, et al. Talimogene laherparepvec (T-VEC) for the treatment of advanced melanoma. Immunotherapy 7, 611-619 (2015)), G207 (Markert, et al. A phase 1 trial of oncolytic HSV-1, G207, given in combination with radiation for recurrent GBM demonstrates safety and radiographic responses. Mol. Ther. 22, 1048-1055 (2014)) and G47Δ (Todo, T. et al. Intratumoral oncolytic herpes virus G47Δ for residual or recurrent glioblastoma: a phase 2 trial. Nat. Med. 28, 1630-1639 (2022)). The strain HSV-2 is also drawing increasing attention and is under investigation at the moment. An oHSV-2 named OH2 has launched phase I/II clinical trials in solid tumors recently, but its modification strategy is the same as that of T-VEC (Zhang, B. et al. Intratumoral OH2, an oncolytic herpes simplex virus 2, in patients with advanced solid tumors: a multicenter, phase I/II clinical trial. J. Immunother. Cancer 9, e002224 (2021)). Thus, in some embodiments, the virus's backbone is an oHSV, optionally T-VEC, G207, or G474.

### B. Pharmaceutical Compositions

The disclosed oncolytic viruses are typically administered to a patient in need thereof in a pharmaceutical composition. Pharmaceutical compositions containing virus may be for systemic or local administration, such as intratumoral. Dosage forms for administration by parenteral (intramuscular (IM), intraperitoneal (IP), intravenous (IV), intra-arterial, intrathecal or subcutaneous injection (SC)), or transmucosal (nasal, vaginal, pulmonary, or rectal) routes of administration can be formulated. In some embodiments, a therapeutic virus is delivered by local injection, for example intracranial injection preferably at or near the tumor site. In a particular embodiment a therapeutic virus is injected directly into the tumor. The compositions can be formulated for and delivered via catheter into the tumor resection cavity through convection-enhanced delivery (CED).

The virus is typically administered to a subject an effective amount, e.g., as discussed elsewhere herein.

Appropriate dosages can be determined by a person skilled in the art, considering the therapeutic context, age, and general health of the recipient. The selected dosage depends upon the desired therapeutic effect, on the route of administration, and on the duration of the treatment desired. Active virus can also be measured in terms of plaque-forming units (PFU). A plaque-forming unit can be defined as areas of cell lysis (CPE) in monolayer cell culture, under overlay conditions, initiated by infection with a single virus particle. Generally dosage levels of virus between 102 and 1012 PFU are administered to humans. In some embodiments, virus is administered in a liquid suspension, in a volume ranging between 10 μl and 100 ml depending on the route of administration. Generally, dosage and volume will be lower for intratumoral injection as compared to systemic administration or infusion. The dose may be administered once or multiple times. When administered locally, virus can be administered to humans at dosage levels between 102 and 108 PFU. Virus can be administered in a liquid suspension, in a low volume. For example, the volume for local administration can range from about 20 nl to about 200 μl. Multiple doses can be administered. In some embodiment, multiple injections are used to achieve a single dose. Systemic or regional administration via subcutaneous, intramuscular, intra-organ, or intravenous administration can have higher volumes, for example, 10 to 100 ml.

The term “pharmaceutically acceptable” means a non-toxic material that does not interfere with the effectiveness of the biological activity of the active ingredients. The term “pharmaceutically-acceptable carrier” means one or more compatible solid or liquid fillers, diluents or encapsulating substances which are suitable for administration to a human or other vertebrate animal. The term “carrier” refers to an organic or inorganic ingredient, natural or synthetic, with which the active ingredient is combined to facilitate the application. Pharmaceutical compositions may be formulated in a conventional manner using one or more physiologically acceptable carriers including excipients and auxiliaries which facilitate processing of the active compounds into preparations which can be used pharmaceutically. The compositions may be administered in combination with one or more physiologically or pharmaceutically acceptable carriers, thickening agents, co-solvents, adhesives, antioxidants, buffers, viscosity and absorption enhancing agents and agents capable of adjusting osmolarity of the formulation. Proper formulation is dependent upon the route of administration chosen. If desired, the compositions may also contain minor amounts of nontoxic auxiliary substances such as wetting or emulsifying agents, dyes, pH buffering agents, or preservatives. The formulations should not include membrane disrupting agents which could kill or inactivate the virus.

In a preferred embodiment, compositions including oncolytic viruses disclosed herein, are administered in an aqueous solution, by parenteral injection. Injection includes, but it not limited to, local, intratumoral, intravenous, intraperitoneal, intramuscular, or subcutaneous injection. The formulation may also be in the form of a suspension or emulsion. In general, pharmaceutical compositions are provided including effective amounts of virus, and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as anti-oxidants (e.g., ascorbic acid, sodium metabisulfite), and preservatives and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. A preferred solution is phosphate buffered saline or sterile saline.

### III. Methods of Treatment

Also provided are methods of using the disclosed oncolytic viruses for treating a subject having a cancer by administering an effective amount of an oncolytic virus to the subject. Some embodiments further include administering the virus in conjunction with at least a first DNA damaging agent, an immunotherapy, another oncolytic virus, and/or another active agent.

### A. Combination Cancer Therapies

In certain embodiments, the compositions and methods of the present embodiments can involve the combination of an oncolytic virus expressing an sCD44, e.g., oHSV-sCD44, alone or in combination with other oncolytic viruses, (for example, oHSV, particularly oHSV-shPKR or Vstat120-expressing oHSV, such as RAMBO). In some embodiments it may also include administering the subject an oncolytic virus expressing an sCD44, e.g., oHSV-sCD44, alone or in combination with at least one DNA damaging agent, such as radiation therapy. In some aspects, the subject may be administered the present combination therapy and an additional anti-cancer therapy, such as a platinum-based DNA damaging agent (e.g., cisplatin, oxaliplatin, and/or carboplatin), see for example, U.S. Patent Publication US20210100859A1, surgery (e.g., lumpectomy and a mastectomy), additional chemotherapy, gene therapy, DNA therapy, viral therapy, RNA therapy, immunotherapy, bone marrow transplantation, nanotherapy, monoclonal antibody therapy, or a combination of the foregoing. The additional anti-cancer therapy may be in the form of adjuvant or neoadjuvant therapy. In particular aspects, the additional anti-cancer therapy may be an immune checkpoint inhibitor, such as a PD-1 or CTLA-4 inhibitor. In some embodiments, the additional anti-cancer therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery.

In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemo preventative agent. The additional therapy may be one or more of the chemotherapeutic agents known in the art. The combination therapy (i.e., oncolytic virus and DNA damaging agent) may be administered before, during, after, or in various combinations relative to an additional anti-cancer therapy, such as immune checkpoint therapy. The administrations may be in intervals ranging from concurrently to minutes to days to weeks. In embodiments where the combination therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the combination therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below the combination therapy of an oncolytic virus and DNA damaging agent is “A” and an additional anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wks), to single doses of 2000 to 6000 roentgens. Dosage ranges for radioisotopes vary widely, and depend on the half-life of the isotope, the strength and type of radiation emitted, and the uptake by the neoplastic cells.

### 2. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include the following though not limited to: alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) include monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4. The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin including an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules. In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody includes the heavy and light chain CDRs or VRs of ipilimumab. Accordingly, in one embodiment, the antibody includes the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. HIF-1α Inhibitors

The results presented below show that the combination oncolytic virus expressing a soluble CD44 and an inhibitor of Hypoxia-inducible factor-1α (HIF-1α) lead to a further reduction in HIF1α, SOX2 and pERK1/2 expression, relative to virus alone. Thus, in some embodiments, the oncolytic virus is administered to the subject in combination with a HIF-1α inhibitor. HIF-1α activity inhibitors can be divided into the following types according to the specific mechanism of action: (1) influence the degradation of HIF-1α; (2) inhibit the DNA transcription and expression of HIF-1α; (3) block the translation of mRNA (4) hinder the binding of HIF-1α and HRE; (5) impede the formation of HIF-1α transcription complex, etc. Examples of HIF-1α inhibitors are known in the art. See, e.g., Xu R, Wang F, Yang H, Wang Z. “Action Sites and Clinical Application of HIF-1α Inhibitors.” Molecules. 2022 May 26; 27(11):3426. doi: 10.3390/molecules27113426. PMID: 35684364; PMCID: PMC9182161, which is specifically incorporated by reference herein in its entirety. Any of the HIF-1α inhibitors mentioned therein, or elsewhere, can be used in combination with the disclosed OV.

### 6. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### B. Subjects to be Treated

In general, the disclosed oncolytic viruses and methods of treatment thereof are useful in the context of cancer, including tumor therapy, particular brain tumor therapy.

In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. The examples below indicate that the viruses and methods disclosed herein are useful for treating cancer, particular brain tumors, in vivo.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. The disclosed compositions are particularly effective in treating carcinomas. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers such as vascular cancer such as multiple myeloma, adenocarcinomas and sarcomas, of bone, bladder, brain, breast, cervical, colo-rectal, esophageal, kidney, liver, lung, nasopharangeal, pancreatic, prostate, skin, stomach, and uterine. In some embodiments, the disclosed compositions are used to treat multiple cancer types concurrently. The compositions can also be used to treat metastases or tumors at multiple locations.

The disclosed methods are particularly useful in treating brain tumors. Brain tumors include all tumors inside the cranium or in the central spinal canal. They are created by an abnormal and uncontrolled cell division, normally either in the brain itself (neurons, glial cells, astrocytes, oligodendrocytes, ependymal cells, myelin-producing Schwann cells, lymphatic tissue, blood vessels), in the cranial nerves, in the brain envelopes (meninges), skull, pituitary and pineal gland, or spread from cancers primarily located in other organs (metastatic tumors). The cancer can be a glioma. Examples of brain tumors include, but are not limited to, glioblastoma, oligodendroglioma, meningioma, supratentorial ependymona, pineal region tumors, medulloblastoma, cerebellar astrocytoma, infratentorial ependymona, brainstem glioma, schwannomas, pituitary tumors, craniopharyngioma, optic glioma, and astrocytoma.

“Primary” brain tumors originate in the brain and “secondary” (metastatic) brain tumors originate from cancer cells that have migrated from other parts of the body. Primary brain cancer rarely spreads beyond the central nervous system, and death results from uncontrolled tumor growth within the limited space of the skull. Metastatic brain cancer indicates advanced disease and has a poor prognosis. Primary brain tumors can be cancerous or noncancerous. Both types take up space in the brain and may cause serious symptoms (e.g., vision or hearing loss) and complications (e.g., stroke). All cancerous brain tumors are life threatening (malignant) because they have an aggressive and invasive nature. A noncancerous primary brain tumor is life threatening when it compromises vital structures (e.g., an artery). In a particular embodiment, the disclosed compositions and methods are used to treat cancer cells or tumors that have metastasized from outside the brain (e.g., lung, breast, melanoma) and migrated into the brain.

The invention can be further understood by the following numbered paragraphs:

1. A modified herpes simplex virus engineered to express the extracellular domain of CD44.

2. The modified herpes simplex virus of paragraph 1, wherein the virus is an oncolytic HSV-1.

3. A pharmaceutical composition for use in treating a subject having a radiation resistant cancer, said composition including an effective amount of the oncolytic virus of paragraph 2.

4. A method of treating a subject have a cancer including administering an effective amount of oncolytic virus of paragraphs 1 or 2 to the subject in conjunction with a at least a first DNA damaging agent.

5. The method of paragraph 4, wherein the DNA damaging agent is DNA-damaging chemotherapeutic agent.

6. The method of paragraph 4, wherein the DNA damaging agent is radiation.

7. The method of paragraph 4, wherein the administration of the oncolytic virus increases tumor cell sensitivity to tumor cell killing.

8 The method of paragraph 4, wherein the administration of the oncolytic virus increases immune cell infiltration of the tumor.

9. The method of paragraph 4, wherein the administration of the oncolytic virus decreases the number of stem cell properties of the cells within in a tumor.

10. The method of paragraph 4, wherein the administration of the oncolytic virus induces DNA damage in a tumor.

11. The method of paragraph 4, wherein the administration of the oncolytic virus downregulates MAPK signaling and inhibits the growth of the tumor.

12. The modified herpes simplex virus of paragraph 2, wherein the administration of the oncolytic virus increases tumor cell sensitivity to a DNA damaging agent.

13. The modified herpes simplex virus of paragraph 12, wherein the DNA damaging agent is radiation.

14. The modified herpes simplex virus of paragraph 12, wherein the DNA damaging agent is a chemotherapeutic agent.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Methods of the Examples

Virus construction, purification and titration: oHSV-sCD44 was constructed using the HSV-Quik protocol as described previously (Terada, et al., Development of a rapid method to generate multiple oncolytic HSV vectors and their in vivo evaluation using syngeneic mouse tumor models, Gene Ther., (8):705-14, PMID: 16421599 (2006), Agarwalla &Aghi, Oncolytic herpes simplex virus engineering and preparation, Methods Mol Biol., 797:1-19, PMID: 21948465 (2016), Russell, et al., PTEN expression by an oncolytic herpesvirus directs T-cell mediated tumor clearance, Nat Commun., 9(1):5006, PMID: 30479334 (2018)). Briefly, soluble CD44 (SEQ ID NO:4) expressed under a transgene (SEQ ID NO:6) driven by the HSV immediate early (IE) promoter IE4/5 was constructed. The cassette was inserted into the disrupted ICP6 locus of an F-strain attenuated HSV bacterial artificial chromosome, driving the expression of sCD44 alongside other viral IE genes. An ICP6/GFP fusion was included to allow viral replication to be traced via fluorescence. BAC DNA was transfected into Vero cells along with the helper plasmids to excise the bacterial origin of replication, facilitating the production of replication competent virus particles. Clonal expansion was achieved through plaque purification and amplification in Vero cells.

Virus purification was done as previously described (Russell, et al., PTEN expression by an oncolytic herpesvirus directs T-cell mediated tumor clearance, Nat Commun., 9(1):5006, PMID: 30479334 (2018)). Virus titers were determined on Vero cells using a standard plaque forming unit (PFU) assay. Briefly, purified virus was serially diluted, added to a 96-well plate and overlaid with 1×10⁴ Vero cells/well and incubated at 37° C. overnight followed by addition of human IgG (1 μg/mL). Plates were incubated at 34° C. for 48-72 h to allow plaque formation. Plaques were counted using fluorescence microscopy and virus concentration was calculated as PFU/mL.

Cell lines: LN229, U87ΔEGFR and Vero cells were maintained in Dulbecco's modified Eagle's medium (DMEM) supplemented with 10% fetal bovine serum (FBS) and 100 IU/mL penicillin, and 100 μg/mL streptomycin. Human glioma neurospheres (GBM12, GBM28 and GBM43) were cultured as tumor spheres as described in (Otani, et al., Oncolytic HSV-Infected Glioma Cells Activate NOTCH in Adjacent Tumor Cells Sensitizing Tumors to Gamma Secretase Inhibition. Clin Cancer Res., 26(10):2381-2392, PMID: 32139403 (2020)). U87ΔEGFR and LN229 human glioblastoma cell lines were verified by short tandem repeat (STR) profiling to match DSMZ-held stocks at all loci on Jan. 14, 2015. All cells are routinely STRS profiled, maintained at a passage below 50, and checked for mycoplasma.

Animals Mouse orthotopic intracranial-injection model: All the animal experiments were conducted with the approval of the University of Texas Health Science Center IACUC and were in compliance with relevant ethical regulations. For the stereotactic intracranial injection, mice were anesthetized with an intraperitoneal injection of 0.2 ml of stock solution containing ketamine HCl (25 mg/ml), xylazine (2.5 mg/ml) dilute in sterile water, stabilized in a stereotactic device (Kopf), and a midline incision was made following sterilization of the surgical site with 70% ethyl alcohol. 2 mm lateral and 2 mm anterior to bregma, a 1 mm burr hole was drilled, and GBM cells or oHSV in 2 ul PBS were intracranially injected using needles (Hamilton 80300 for cells or Hamilton 80000 for virus) at a depth of 3 mm and at a rate of 0.4 μl/min using autoinjectors (KD Scientific). Needles were removed, and the skin was sutured using nylon thread. For survival studies, 5×10⁴ DB7 breast cancer or U87ΔEGFR cells were implanted into the brains of 6-8 week old immunocompetent FVB/N (Jackson Labs) or immunocompromised NSG mice, respectively. Seven days after tumor cell implantation, mice were treated intratumorally with sterile saline control, or 1e5 PFU of either HSV-ctl or oHSV-sCD44.

qPCR: Total RNA from cell culture was isolated using Qiagen RNeasy Mini Kit (Qiagen) as per the manufacturer's instructions. For cDNA synthesis, 2 μg of quantified RNA was reverse transcribed with High Capacity cDNA Reverse Transcription Kit (Applied Biosystems, Foster City, CA, USA) using random hexamer. Primers for qPCR were designed using Primer3 software or referred from literature (Table 1) and synthesized from IDT company. qPCR was performed using qPCRBIO SyGreen Mix Lo-ROX (Genesee Scientific, CA, USA) in QuantStudio™ 3 Real-Time PCR System (Thermo Fisher Scientific, Waltham, MA, USA) in 96-well format with the following cycling conditions: Initial denaturation 95° C.-2 mins; amplification cycle: denaturation 95° C.-15 s, annealing 60° C.-20 s and extension 72° C.-5 s. Amplicon specificity was confirmed by melting curve analysis. Tubulin and 18S rRNA were used as housekeeping controls and 2(Act) method was utilized to calculate the differential fold change in the expression.

Western Blot Analysis: Protein isolation and Western blotting procedures have been described before (Russell, et al., PTEN expression by an oncolytic herpesvirus directs T-cell mediated tumor clearance, Nat Commun., 9(1):5006, PMID: 30479334 (2018)). Briefly, cultured cells infected with either HSVctl or oHSV-sCD44 or saline at the indicated MOIs were harvested 48 hpi in RIPA cell lysis buffer with protease and phosphatase inhibitors added prior to use. Lysates were sonicated, cleared by centrifugation and total protein was quantified by BCA (Thermo Fisher Scientific, Waltham, MA, USA). Equal amount of lysates were resolved in 4-20% precast gels, transferred onto PVDF membranes and probed with primary antibodies following manufacturer's protocol. All antibody incubations were performed overnight at 4° C. HRP-conjugated secondary antibodies and ECL Prime Western Blotting Detection Reagent (Amersham, USA) were used for protein band detection using ChemiDoc MP Imaging System (BioRad). Anti-beta tubulin antibody was used as protein loading and transfer control.

Immunofluorescence (IF) Studies: GBM cells were plated on chamber slides pre-coated with GelTrex and treated with HSV-ctl or HSV-sCD44. 48 h post treatment, cells were fixed in 4% paraformaldehyde for 10 minutes and permeabilized with 0.01% Triton-X in PBS for 5 minutes. The cells were blocked with 10% goat serum in PBS-0.05% TritonX-100 (PBST) for 1 hr prior to incubation with primary antibodies diluted in 1% goat serum. The slides were washed three times with PBST and cells were incubate with the secondary antibody for 1 hr. DAPI was used for staining cell nuclei. The slides were washed, mounted using Fluormount-G (Thermo Fisher Scientific) and analyzed under confocal microscope.

Immunohistochemistry (IHC) Studies: Tumor tissues were fixed and embedded in paraffin and sections were prepared. After deparaffinization and antigen retrieval in sodium citrate buffer (pH 6.0) at 90° C. for 30 mins, the tissue sections were permeabilized with 0.01% Triton-X in PBS for 5 minutes followed by blocking with 10% goat serum in PBS-0.05% Triton-20 (PBST) for 1 hr. Sections were incubated overnight in primary antibody at a dilution of 1:100 in 1% goat serum, washed three times with PBST, incubated in fluorophore conjugated secondary antibody (1:250) for 1 hr, washed and mounted using Flouormount-G mounting media. DAPI was used for nuclear staining. Slides were analyzed under confocal microscope (Nikon, Japan).

Reporter assay: For HIF-1a reporter assay, GBM cells were transfected with negative control or HIF-1α transcription response element linked to a firefly luciferase reporter gene (System Biosciences, CA, USA) using Lipofectamine reagent (Thermo Scientific, USA) 48 h before infection. Luciferase activity was assayed using Firefly Luciferase Reporter Assay System (Promega, USA) 24 h and 48 h post-infection. Luciferase activity was measured with BioTek Multi-Mode Microplate Reader (Biotek, CA, USA).

Flow cytometry analysis: immune response: Whole brain was harvested 3^{rd} and 7^{th} day post virus injection and tumor bearing hemisphere was processed for immune profile analysis using the tumor cell dissociation kit and debris removal kit as per manufacturer's instruction. Cells were washed with PBS and blocked with Fc blocker (BD Biosciences) for cell surface staining. Fluorochrome-labeled antibodies (Annexin-V, CD45, CD11c, CD4, CD8, CD11b, Ly6G, Ly6C, PD-1, PD-L1, F4/80, CD56, CD86, HLA-DR, CD206, CD44) purchased from BD Biosciences were added to the cells, and stained for 30 minutes as described previously. For intracellular staining, cells were permeabilized with Fix/Perm buffer (FC009, R&D systems) for 20 minutes and then washed with Perm/Wash buffer (R&D systems). Fluorochrome-labeled antibody Foxp3 (560408, BD Biosciences) were diluted in Perm/wash buffer and stained for 30 minutes as described previously. All samples were analyzed on a CytoFlex flow cytometer (Beckman Coulter).

Soft agar colony formation assay: Six-well plates were pre-coated with 1% agarose in 5% FBS containing DMEM. GBM cells were trypsinized and 1×10⁴ cells/well were suspended in DMEM containing 20% FBS, 4 mM glutamine and 2% penicillin/streptomycin with 0.35% agarose and seeded on to the pre-coated six-well plates. Cells were replenished with 200 μl media every 2 days and allowed to form colonies for 21 days. ImageJ software (NIH, USA) was used to calculate the number and area of tumorspheres, area represented as arbitrary units (AU).

RNA library and data analysis: For RNA sequencing (RNA-seq), total RNA was prepared from GBM28 cells treated with saline/oHSVctl/oHSV-sCD44 (MOI 0.005) with or without IR (2×2Gy) 48 hr post infection. Total RNA was extracted utilizing RNeasy Mini Kit (74104, Qiagen), Poly (A)-tailed mRNA was enriched and the RNA-seq library was constructed by the UTHealth Cancer Genomics Core following the manufacturer's instructions for the KAPA mRNA HyperPrep Kit (KK8581, Roche Holding AG) and the KAPA Unique Dual-indexed Adapter Kit (KK8727, Roche Holding AG). RNA-seq data were generated by an Illumina Nextseq 550 using the 75 bp pairended running mode. Raw mRNA sequence reads were preprocessed using Cutadapt (v1.15) to remove bases with quality scores<20 and adapter sequences. Clean RNA-seq reads were aligned to the reference genome GRCh38.83 with STAR (v2.5.3a). Gene abundance was measured by HTseq-count uniquely mapped read number with default parameters and using annotations from ENSEMBL v83. Only the genes with >5 reads in at least one sample were used for differential expression analysis by DESeq2 software, which implements a negative binomial distribution model. Resulting P values were adjusted using the Benjamini and Hochberg approach to control for FDR. Genes with FDR<0.05 were considered as differentially expressed (DE) for follow-up analysis. Gene set enrichment analysis (GSEA) was conducted using RDAVID WebService (v1.19.0) for Gene Ontology (GO) terms and R package fgsea for Kyoto Encyclopedia of Genes and Genomes (KEGG) pathway analysis, respectively. The enrichment P values were adjusted by following Benjamini and Hochberg approach.

Statistical analysis. Statistical analyses were performed using GraphPad Prism (GraphPad, San Diego, CA). Student's t-test or Mann-Whitney U test were used to test the difference in comparison of continuous data between the two groups. One way and two way analysis of variant (ANOVA) was used to evaluate the difference between more than 2 groups and more than one categorical variable. To test the association between categorical data, a contingency table was made and Chi-square or Fisher's Exact test was used to evaluate the statistical significance. To analyze survival data, Kaplan-Meier curves were compared using the log-rank test and the post hoc pairwise groups test (if applicable) was further performed by Benjamini and Hochberg correction. P-values≤0.05 were considered statistically significant.

Limited dilution assay. Limited dilution assay was performed as described earlier by Agro and O'Brien (Agro & O'Brien, In vitro and in vivo Limiting Dilution Assay for Colorectal Cancer, Bio Protoc., 5(22):1-11, PMID: 29468185 (2015)). Briefly, 5×10⁵ GBM cells/well were seeded in a 6-well plate and cultured for 24 h followed by infection with oHSVQ or oHSV-sCD44 at an MOI of 0.005. 48 hr post infection cells were trypsinized and collected by centrifugation at 200 g for 5 mins. Cell pellet was resuspended in 5 ml media, cells were counted and diluted in serum free cell culture media (DMEM/F-12 supplemented with 1% Penicillin-streptomycin, 2 mM L-glutamine, 1× Non-essential amino acids, 1 mM Sodium Pyruvate, 1×HEPES, 4 μg/ml Heparin, 20 ng/ml EGF, 10 ng/ml bFGF and 1×N2 supplement-A) to get a final concentration of 1×10⁵ cells/ml. Cells were then further serially diluted to get the desired concentrations of 1000, 100, 10 and 1 cells/well in a 96-well plate. Each concentration was seeded in a replicate of 8 per group. 10 days after seeding, bright field images were captured using Invitrogen EVOS microscope and enriched and depleted stem cell populations were analyzed using the Extreme Limited Dilution Analysis (ELDA) Web tool (Hu & Smyth, ELDA: extreme limiting dilution analysis for comparing depleted and enriched populations in stem cell and other assays, J Immunol Methods, 347(1-2):70-8, PMID: 19567251 (2009)) and were represented as log fraction nonresponding.

### Example 1: CD44 is Expressed in Both GBM and GSC In Vitro and in Vivo

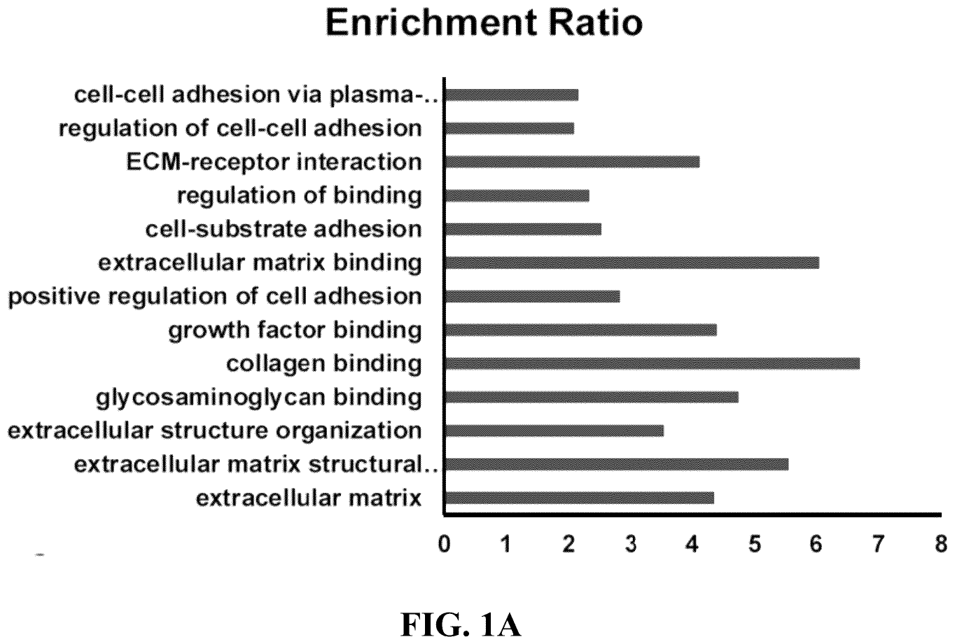

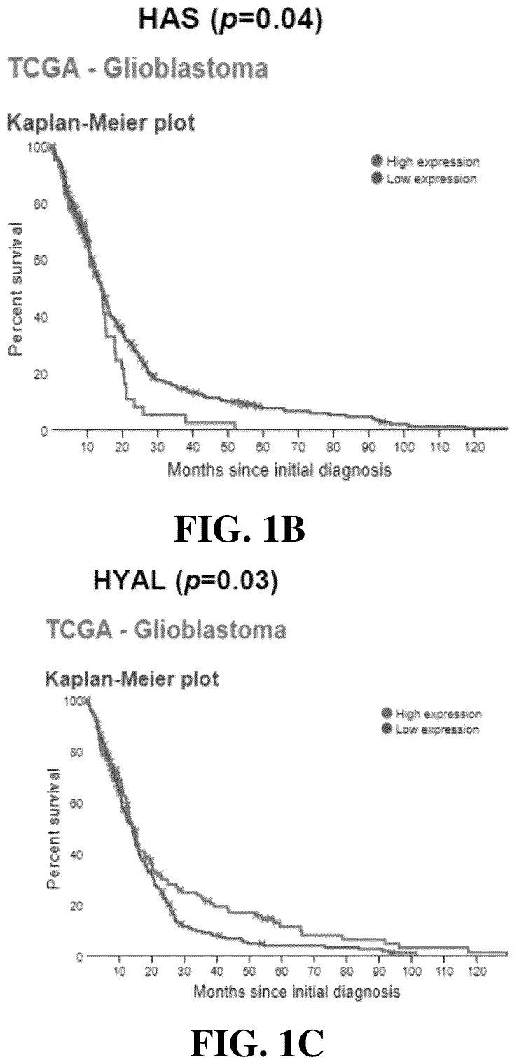

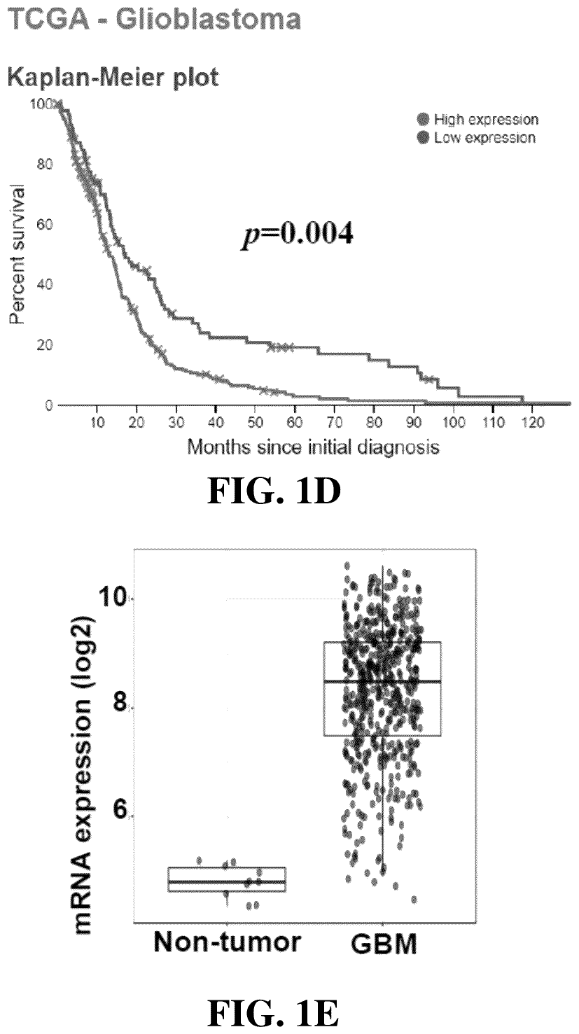

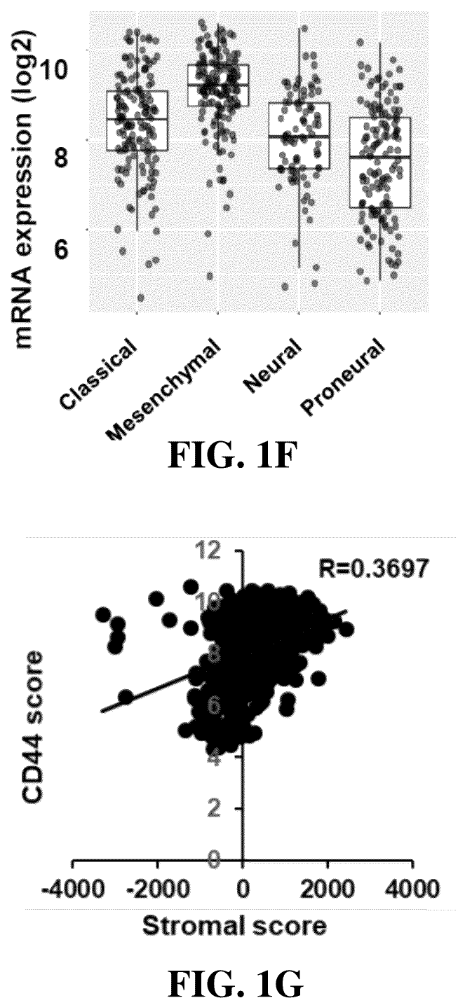

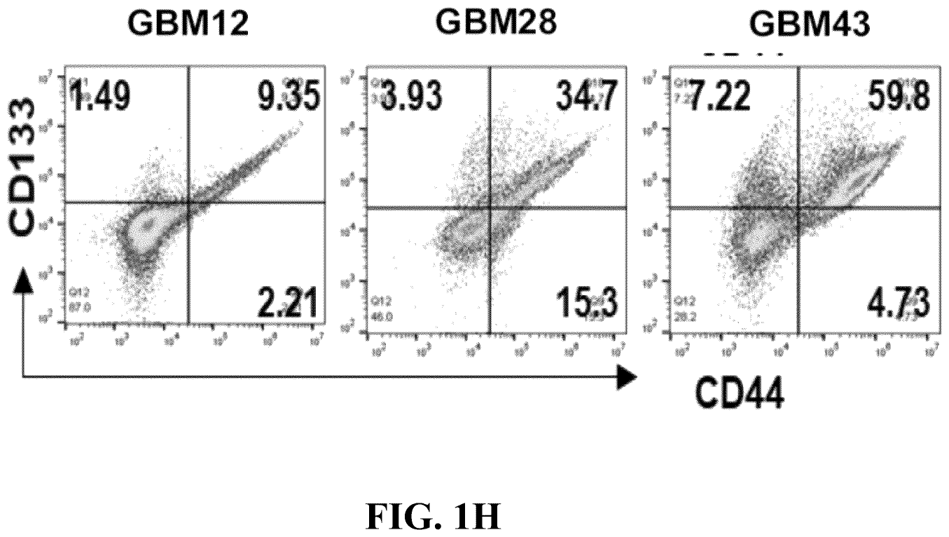

Oncolytic HSV therapy is being increasingly used for treatment of Cancer. To evaluate changes in tumor cell biology with oncolytic HSV therapy total RNA sequencing of glioma cells treated with oHSV was performed. RNA sequencing of glioma cells revealed an enrichment of pathways related to tumor and extracellular matrix interactions (FIG. 1A). Since the brain is enriched in hyaluronan the impact of oHSV therapy on HA was evaluated in vivo. Brain sections from mice with intracranial tumors were stained for the presence of HA using a HA binding protein. Immunohistochemistry revealed a significant induction of HA in tumors treated with oHSV therapy (data not shown). HA is one of the primary components of the ECM of GBM creating a unique environment that is recognized by tumor cell surface CD44 that transmits cellular signaling which contributes towards the aggressive nature of primary and secondary brain tumors. Consistent with this, TCGA analysis for changes in gene expression of Hyaluronan Synthase (HAS, enzyme that makes hyaluronan) and Hyaluronidase (HYAL, key HA degrading enzyme) showed that patients with higher HAS had a worse prognosis and patients with a higher amount of HYAL had a better prognosis (FIGS. 1B-1C). Kaplan-Meier survival curves from TCGA-dataset of grade IV primary tumors around 400 patients further showed that higher CD44 expression correlated with a worse prognosis for GBM patients (FIG. 1D). Significantly higher levels of CD44 gene expression are also observed in tumor tissue derived from GBM patients compared to normal brain (FIG. 1E), Among GBM transcriptional subtypes, CD44 was observed to have the highest expression in the mesenchymal subtype (FIG. 1F). CD44 expression is known to increase tumor stem cell stemness and consistent co-staining of CD44 with stem cell marker CD133 in GBM neurosphere cultures (FIG. 1H) Since CD44/HA signaling is associated with tumor stemness this implied that the residual tumor after oncolysis might be enriched for a tumor stem cell population. Analysis of TCGA database also revealed a significant correlation between CD44 expression and stromal scores of patients (FIG. 1G). Stromal score is generated using an algorithm-Estimation of Stromal and Immune cells in Malignant Tumors using Expression data (ESTIMATE) to analyze TCGA data sets, which include 141 genes. ESTIMATE predicts the purity and immune/stromal cell infiltration in various tumors. Stromal cells are involved in disease progression and resistance in various tumor types and was developed to assist as a model for prognosis, and it has been shown that higher stromal scores are associated with worse outcomes in patients diagnosed with GBM.

### Example 2: oHSV-CD44 Secretes CD44 from GBM and Increase Tumor Lysis

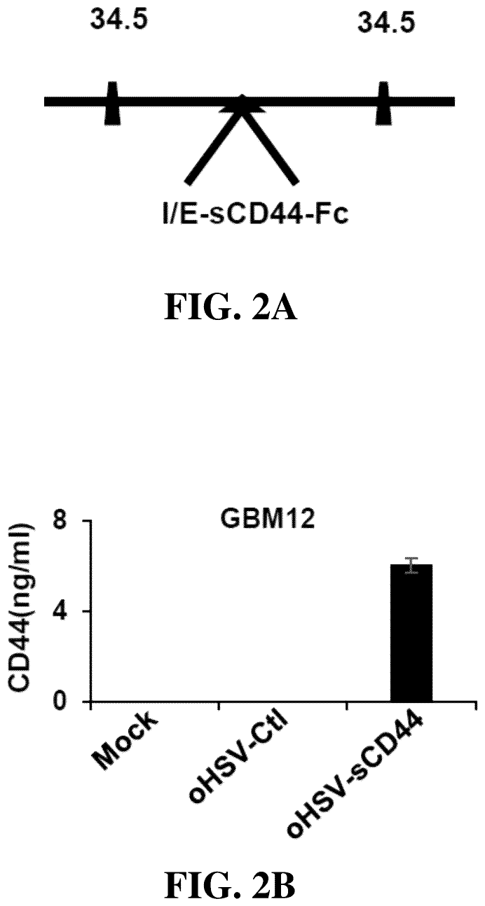

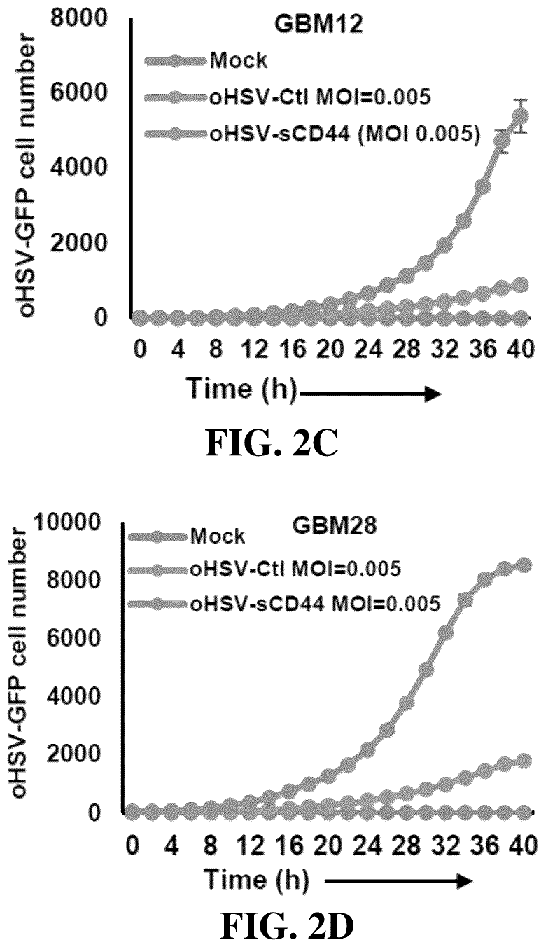

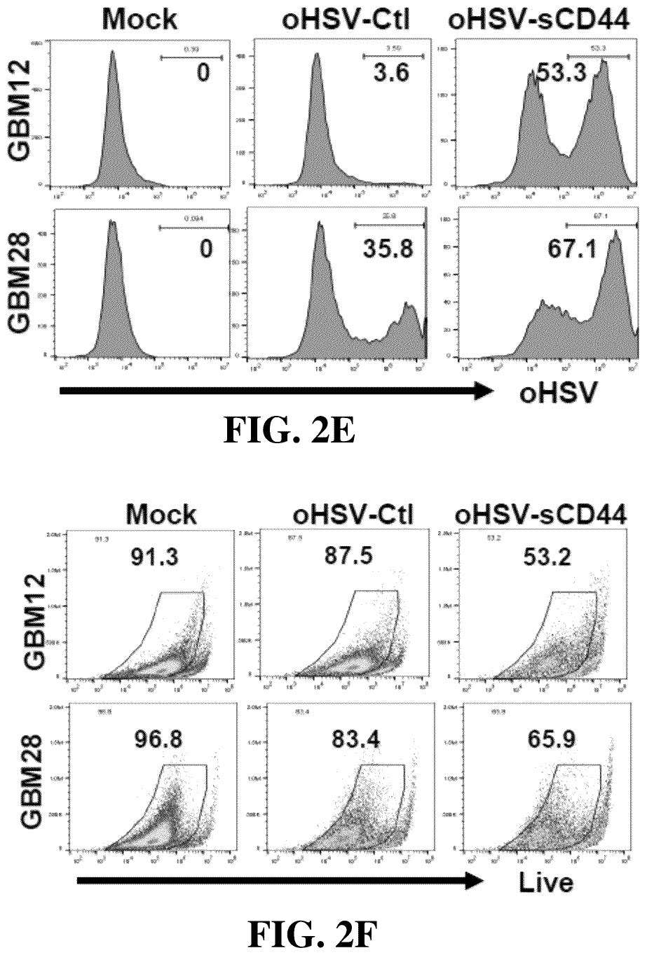

GBM stem cells represent a subset of cells that are resilient to standard therapy. Increased tumor cell stemness along with increased HA after virotherapy implied that CD44/HA signaling presented a significant barrier to anti-tumor efficacy of oHSV therapy. To evaluate if blocking CD44 signaling would affect virotherapy an oncolytic virus was engineered that encoded for truncated extracellular CD44 (sCD44) that can function as a dominant negative receptor to block signaling mediated by standard CD44 receptor. FIG. 2A illustrates the genetic structure of oHSV-sCD44: an oHSV encoding for sCD44 backbone. Western blot (data not shown) and ELISA (FIG. 2B) of conditioned medium of infected glioma cells revealed an efficient secretion of sCD44 in the conditioned medium of oHSV-sCD44 treated cells. To test whether sCD44 expression affected viral replication and/or tumor cell killing in vitro, the primary GBM cells were infected with either a control oHSV (oHSV-ctl) control or oHSV-sCD44 and virus infectivity was monitored by quantifying the spread of GFP +ve infected cells over time by live cell fluorescent imaging using Cytation 5 Cell Imaging Multi-Mode Reader connected to a BioSpa 8 Automated Incubator (BioTek Instruments, INC.). oHSV-sCD44 had a significant increase in the kinetics and spread of virus replication compared to control oHSVctl (FIGS. 2C-2D). Consistent with this, flow cytometric analysis for GFP⁺ cells and live dead staining also revealed better infection (FIG. 2E) and increased tumor cell lysis (FIG. 2F) by oHSV-sCD44 as compared to oHSVctl in both GBM12 and GBM28 cells.

### Example 3: Reduced Tumor Cell Stemness after oHSV-sCD44 Treatment

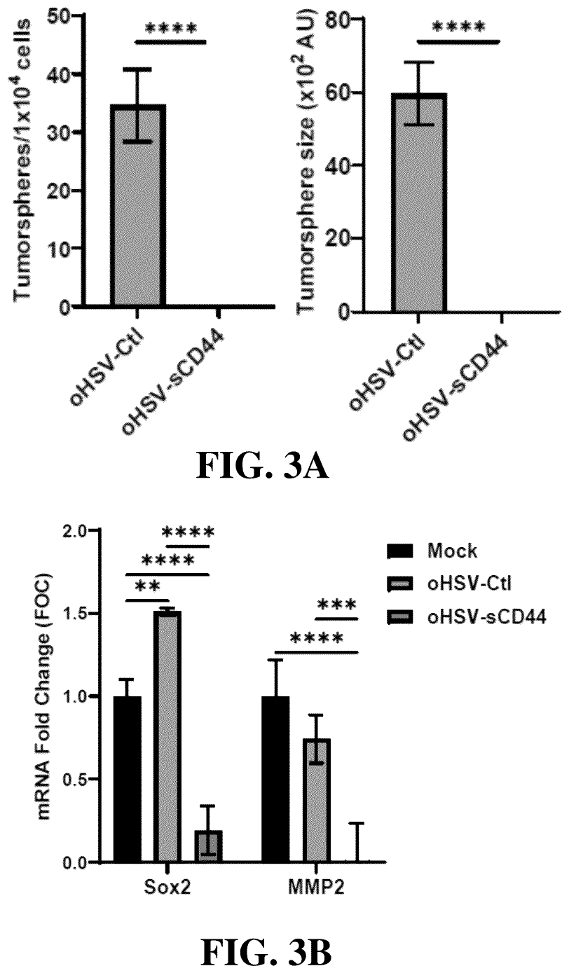

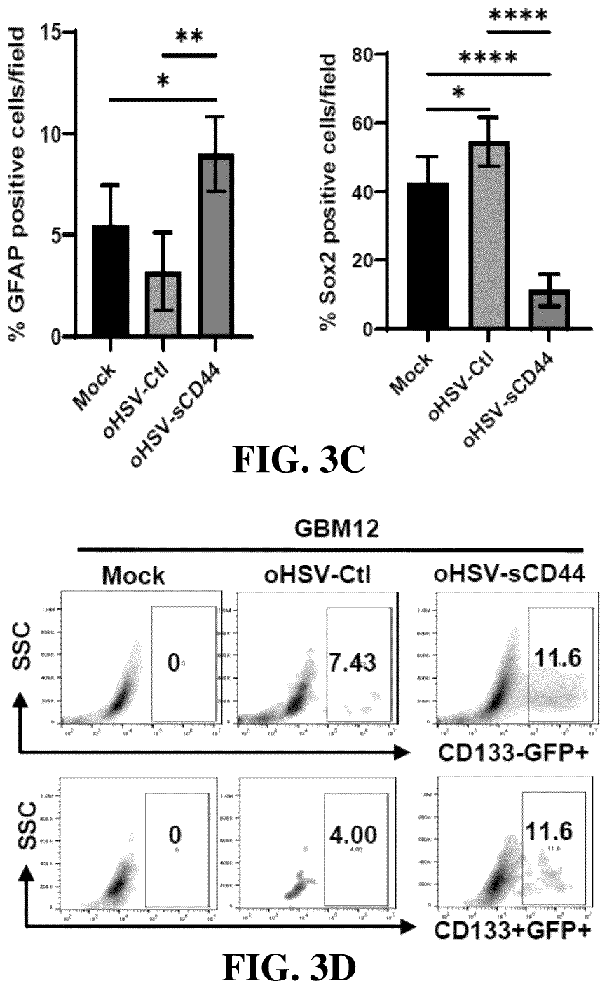

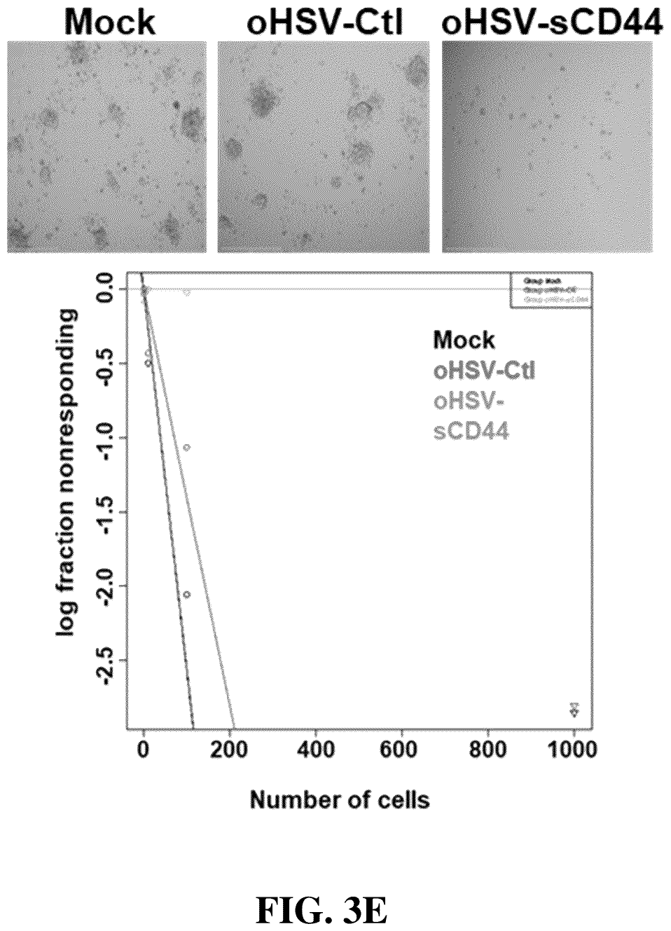

To investigate the impact of oHSV secreted sCD44 on tumor initiating stem cell population, a colony formation assay was performed to detect the ability of infected cells to grow and form colonies in an anchorage independent manner. oHSV-sCD44 treated cells showed an almost complete abrogation in the ability of GBM neurosphere cells to form colonies after treatment with oHSV-sCD44 treatment (FIG. 3A). The lack of colony formation was accompanied by reduced transcript levels of Sox2 and MMP2 (FIG. 3B). Decreased stemness was further confirmed by immunostaining which demonstrated a decreased expression of stem cell marker Sox2, and an increase in the expression of differentiation marker GFAP (FIG. 3C). Immunostaining of tumor sections from GBM bearing mice treated with oHSVctl or oHV-sCD44 also showed increased tumor cell death (Cleaved Caspase 3), reduced tumor growth (Ki-67) and reduced stemness (CD133), and increased differentiation (GFAP) in vivo (data not shown). To further investigate the impact of oHSV secreted sCD44 on GBM tumor initiating stem cell population, an in vitro limited dilution assay that ascertains the clonogenic frequency of uninfected or oHSV-Ctl or oHSV-sCD44 infected primary GBM cells was performed. oHSV-sCD44 infected cells showed a significant reduction in the ability of GBM neurospheres to form colonies (FIG. 3E). While these results show that oHSV-sCD44 reduced the number of glioma stem cell in vitro and in vivo: next evaluated was if there was a difference in the susceptibility of CD133+ve stem cells sensitivity to oHSV treatment. Flow cytometry analysis showed that while oHSV preferentially infected CD133−ve cells, oHSV-sCD44 treatment was equally sensitive to CD133+ve and CD133−ve tumor cells (FIG. 3D).

### Example 4: oHSV-sCD44 Increases Anti-Tumor Efficacy In Vivo

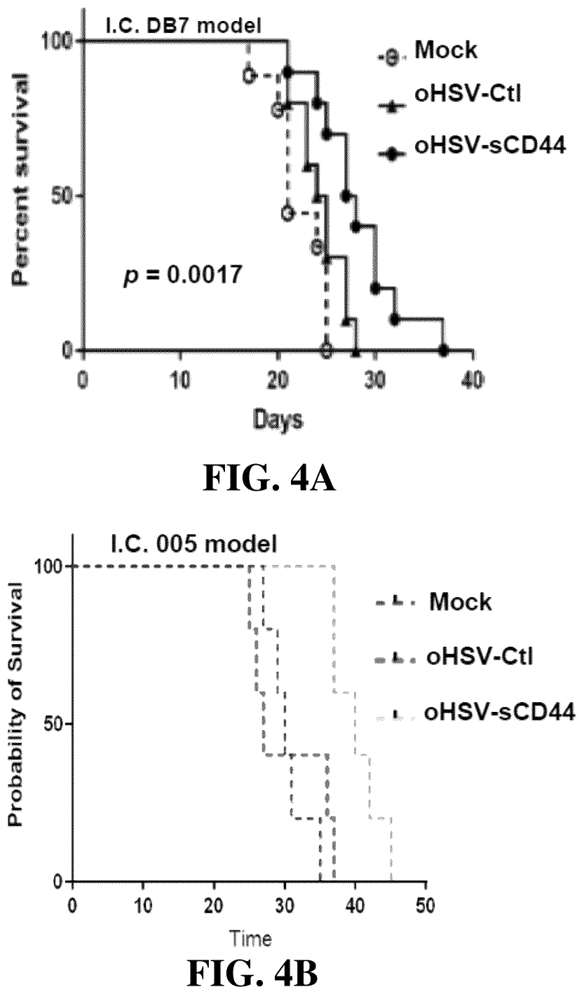

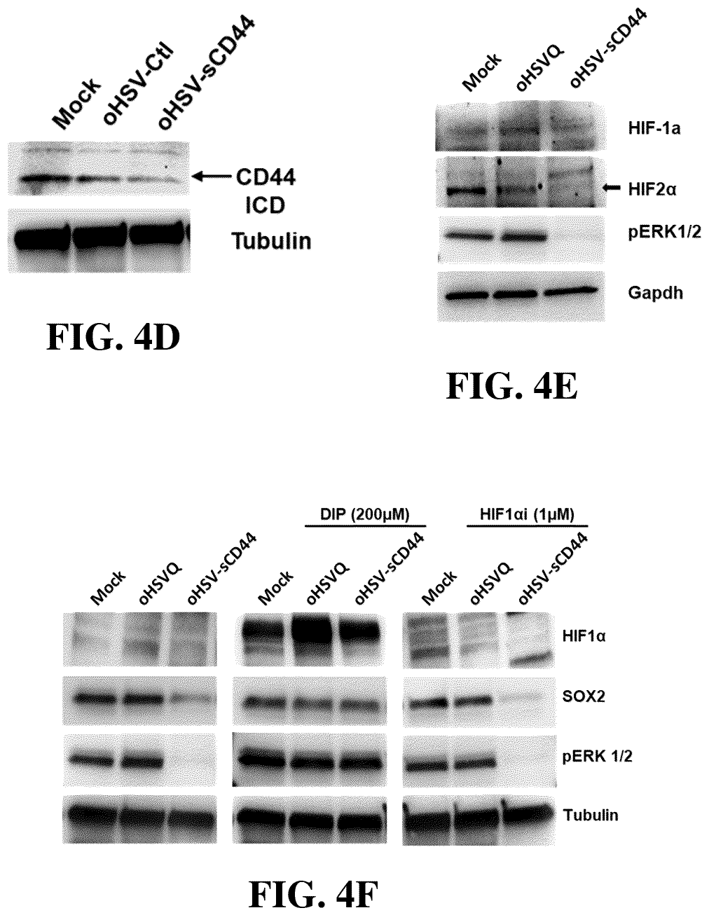

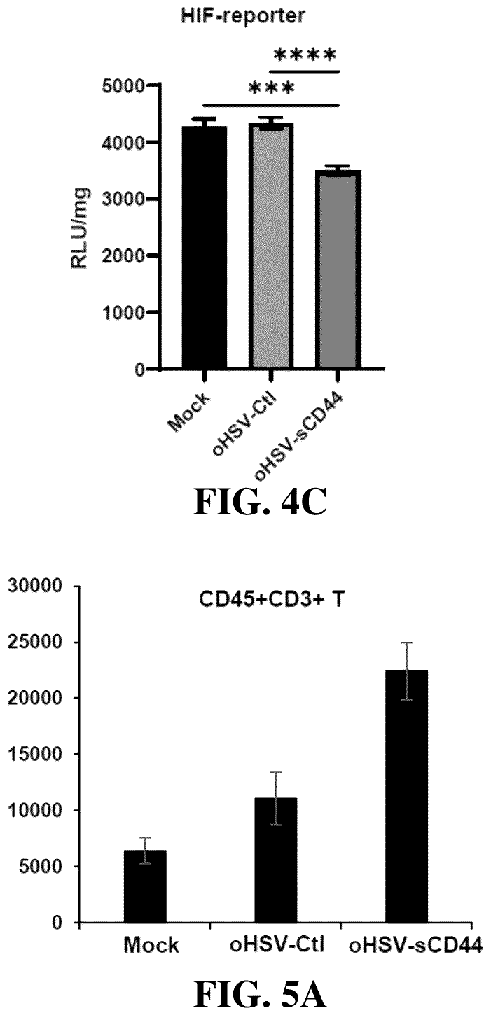

Next investigated was whether oHSV-sCD44 infection of GBM cells could suppress GBM tumor growth and impact survival. The impact of oHSV-sCD44 on antitumor efficacy was tested in intracranial murine 005 model and DB7 breast cancer brain metastases model. To evaluate the impact of oHSV-sCD44 on antitumor efficacy, tumor-bearing mice were treated with a single dose of control oHSV-ctl or oHSV-sCD44. A significant improvement in overall survival of mice treated with oHSV-sCD44 was observed compared to oHSV-ctl treated mice (FIGS. 4A-4B). Moreover, oHSV-sCD44 treated GBM tumors showed a significant reduction in the HA as revealed by reduced HABP binding peptide (HABP) compared to saline or oHSV-ctl treated tumors (data not shown), indicting that sCD44 also interrupted the feed forward CD44 mediated induction of HA synthesis through PI3K signaling. CD44 can mediate intracellular signaling via activation of AKT and MAPK pathway. Upon ligation it is also processed by gamma secretase enzyme to release its intracellular C-terminal domain (CD44ICD) that then translocases to the nucleus and partners with HIF 1/2 to initiate transcription of downstream target genes. HIF Reporter analysis using a HIF responsive promotor driven luciferase also showed that oHSV-sCD44 reduced HIF activity (FIG. 4C).

CD44 mediates intracellular signaling via activation of AKT and MAPK pathway. Upon ligand binding it is processed by gamma secretase enzyme to release its intracellular C-terminal domain (CD44-ICD) which then translocates to the nucleus and partners with HIF 1/2 to initiate transcription of downstream target genes. Western blot analysis showed reduction in CD44-ICD generation in primary GBM cells infected with oHSV-sCD44 as compared to oHSV-Ctl infected cells (FIG. 4D). Immunofluorescence staining further confirmed inhibition of CD44-ICD generation by oHSV-sCD44 infection (data not shown). Western blot analysis of GBM cell lysates treated with oHSV-Ctl or oHSV-sCD44 uncovered that expression of sCD44 by an oHSV rescued HIF 1/2 stabilization and blocked MAPK activation (FIG. 4E). To further understand the involvement of HIFs in oHSV-sCD44-induced reduction in GBM stemness, GBM cells were infected with oHSV-sCD44 in presence or absence of 2,2-dipyridyl (DIP, hypoxia-mimetic to stabilize HIFs), or HIF1α inhibitor (HIF1αi) and checked for the downstream targets of CD44 signaling 48 hr post infection. As seen in FIG. 4F, inhibition of HIF1α synergized with oHSV-sCD44 with further reduction in HIF1α, SOX2 and pERK1/2. Stabilizing HIFs via DIP treatment rescued GBM cells from oHSV-sCD44-induced downregulation of Sox2 and pERK1/2.

### Example 5: oHSV-CD44 Intratumor Injection Induces Immune Cell Infiltration

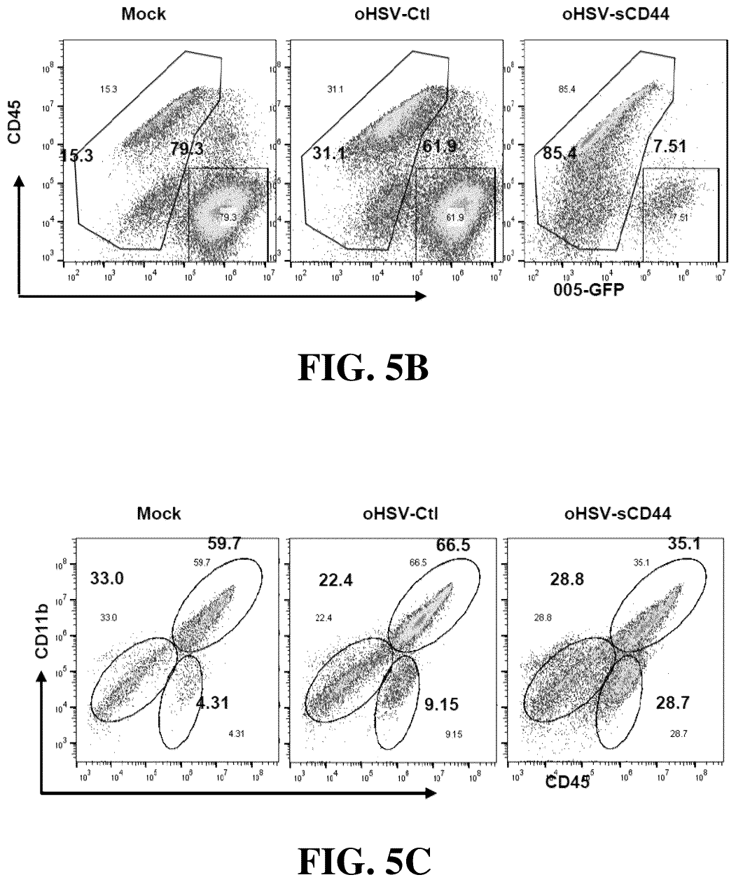

Next analyzed was whether oHSV-CD44 intra-tumor injection affects anti-tumor immune response. tSNE analysis of brains of mice bearing DB7 tumors showed that oHSV-CD44 intra-tumor injection increased both CD4 and CD8 T cell infiltration (data not shown). Detailed flow cytometry analysis revealed that oHSV-sCD44 intra-tumoral injection significantly increased the total cells number of T cells infiltration (FIG. 5A). Flow cytometry analysis of Immune profiles in 005 model also showed a significant reduction in 005 tumor cells while immune cells infiltration was robustly increased (FIG. 5B). Further analysis showed a reduction in CD11b+CD45+ cells with concurrent increase in CD45+CD11b− lymphocytes significantly (FIG. 5C).

### Example 6: oHSV-CD44 Induces DNA Damage in GSC and GBM Neurospheres

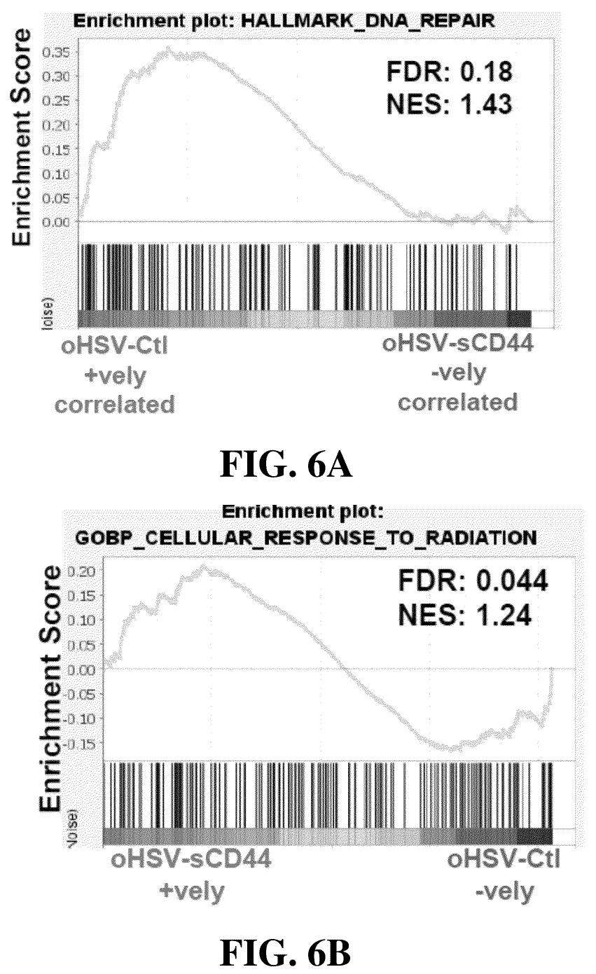

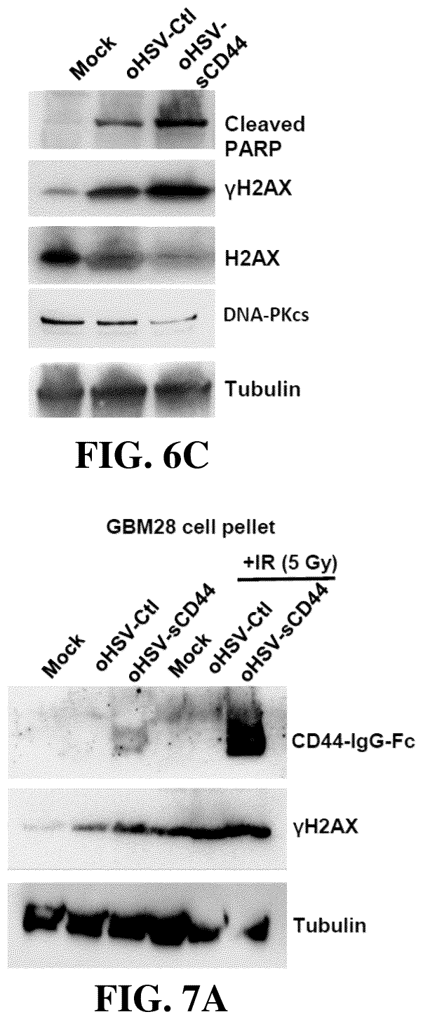

CD44 signaling is associated with radiation resistance. Next evaluated was the impact of CD44 on DNA damage response. Total RNA sequencing of GBM 28 glioma cells infected with oHSV-ctl or oHSV-sCD44 showed a significant reduction in DNA repair pathways and cellular response to radiation (FIGS. 6A-6B, and data not shown). Western blot (FIG. 6C) and immunofluorescence analysis (data not shown) also showed increased phosphorylation of H2Ax and DNA-PKC indicating increased DNA damage in cells treated with oHSV-sCD44 relative to HSV Ctrl-treated cells.

### Example 7: A Combination oHSV-sCD44 with Irradiation Decreases GSC Stemness

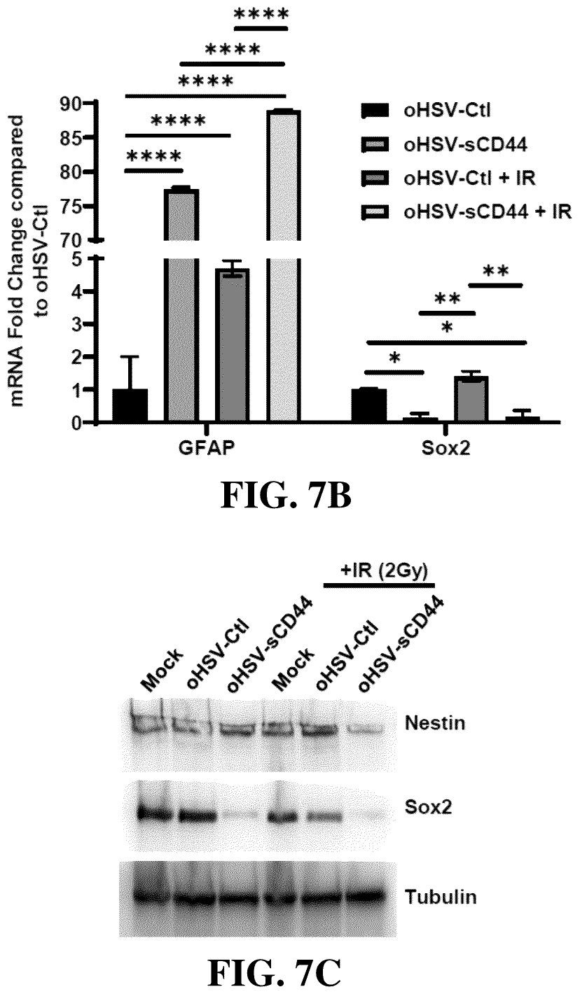

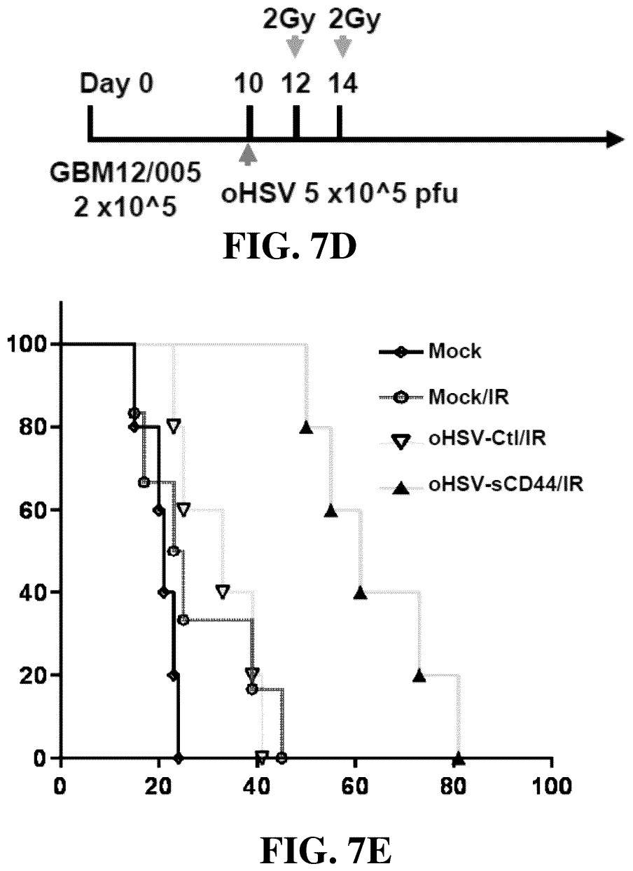

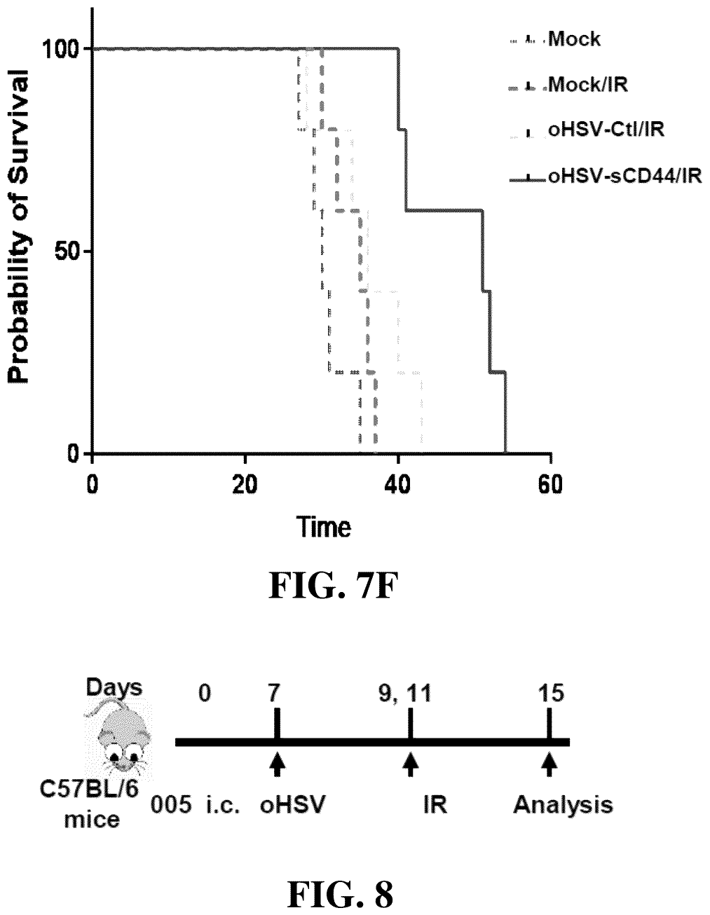

Reduced stemness, increased DNA damage and impaired DNA damage repair implied that oHSV-sCD44 infected glioma cells would be sensitized to radiation. To evaluate the effect of radiation on sCD44 secretion by the infected cells, CD44 expression was investigated with or without exposure to radiation following infection. Radiation did not hamper sCD44 secretion by the infected cells (FIG. 7A). Increased evidence of DNA damage was also observed by increased phosphorylation of H2AX (γH2AX) in cells treated with oHSV-sCD44 and radiation expression induced by radiation (FIG. 7A). A significant reduction in stemness markers Sox2 and Nestin was also observed (FIGS. 7B-7C). Next tested was whether a combination of oHSV-sCD44 with irradiation increases anti-tumor efficacy in vivo. Mice with human GBM12 or murine 005 glioma cells implanted intracranially were treated with oHSV-Ctr or oHSV-sCD44 with irradiation as shown in schema (FIG. 7D) and survival of mice was monitored. FIGS. 7E-7F show Kaplan-Meier curves of survival of these mice. Results showed that the combination significantly prolonged tumor-bearing mice survival in both GBM-12 and 005 model (FIGS. 7D-7F).

### Example 8: Anti-Tumor Efficacy of oHSV-sCD44 is Increased with Irradiation

Immunofluorescence of brain sections of immune competent mice bearing intracranial 005 GBM treated with oHSV and irradiation as shown in the schema (FIG. 8). CD8+ve immunofluorescent staining showed a significant increase in CD8+ cells in tumors treated with the combination of oHSV-sCD44 and irradiation (data not shown). Immune profile analysis by flow cytometry further confirmed that the combination of oHSV-CD44 with irradiation significantly promoted infiltration of CD8+ T cells and CD4+ T cells (data not show).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, and others cited elsewhere herein, to the extent that they provide exemplary procedural or other details supplementary to those set forth herein, are specifically incorporated herein by reference.
ADDIN EN.REFLIST 1. Wesseling, P. and D. Capper, WHO 2016 Classification of gliomas. Neuropathol Appl Neurobiol, 2018. 44(2): p. 139-150.
2. Noch, E. K., R. Ramakrishna, and R. Magge, Challenges in the Treatment of Glioblastoma: Multisystem Mechanisms of Therapeutic Resistance. World Neurosurg, 2018. 116: p. 505-517.
3. Thakkar, J. P., et al., Epidemiologic and molecular prognostic review of glioblastoma. Cancer Epidemiol Biomarkers Prev, 2014. 23(10): p. 1985-96.
4. Martikainen, M. and M. Essand, Virus-Based Immunotherapy of Glioblastoma. Cancers (Basel), 2019. 11(2).
5. Martin, T. A., et al., The role of the CD44/ezrin complex in cancer metastasis. Crit Rev Oncol Hematol, 2003. 46(2): p. 165-86.
6. Ponta, H., L. Sherman, and P. A. Herrlich, CD44: from adhesion molecules to signalling regulators. Nat Rev Mol Cell Biol, 2003. 4(1): p. 33-45.
7. Murai, T., Lipid Raft-Mediated Regulation of Hyaluronan-CD44 Interactions in Inflammation and Cancer. Front Immunol, 2015. 6: p. 420.
8. Ahrens, T., et al., Soluble CD44 inhibits melanoma tumor growth by blocking cell surface CD44 binding to hyaluronic acid. Oncogene, 2001. 20(26): p. 3399-408.
9. Sy, M. S., Y. J. Guo, and I. Stamenkovic, Inhibition of tumor growth in vivo with a soluble CD44-immunoglobulin fusion protein. J Exp Med, 1992. 176(2): p. 623-7.
10. Chen, Y., et al., The prognostic value of CD44 expression in gastric cancer: a meta-analysis. Biomed Pharmacother, 2014. 68(6): p. 693-7.
11. Jin, L., et al., Targeting of CD44 eradicates human acute myeloid leukemic stem cells. Nat Med, 2006. 12(10): p. 1167-74.
12. Marangoni, E., et al., CD44 targeting reduces tumour growth and prevents post-chemotherapy relapse of human breast cancers xenografts. Br J Cancer, 2009. 100(6): p. 918-22.
13. Senbanjo, L. T. and M. A. Chellaiah, CD44: A Multifunctional Cell Surface Adhesion Receptor Is a Regulator of Progression and Metastasis of Cancer Cells. Front Cell Dev Biol, 2017. 5: p. 18.
14. Chen, C., et al., The biology and role of CD44 in cancer progression: therapeutic implications. J Hematol Oncol, 2018. 11(1): p. 64.
15. Sneath, R. J. and D. C. Mangham, The normal structure and function of CD44 and its role in neoplasia. Mol Pathol, 1998. 51(4): p. 191-200.
16. Phillips, H. S., et al., Molecular subclasses of high-grade glioma predict prognosis, delineate a pattern of disease progression, and resemble stages in neurogenesis. Cancer Cell, 2006. 9(3): p. 157-73.
17. Wang, Q., et al., Tumor Evolution of Glioma-Intrinsic Gene Expression Subtypes Associates with Immunological Changes in the Microenvironment. Cancer Cell, 2017. 32(1): p. 42-56.e6.
18. Russell, L., et al., PTEN expression by an oncolytic herpesvirus directs T-cell mediated tumor clearance. Nat Commun, 2018. 9(1): p. 5006.
19. Semenza, G. L., Hypoxia-inducible factors: mediators of cancer progression and targets for cancer therapy. Trends Pharmacol Sci, 2012. 33(4): p. 207-14.
20. Li, Z., et al., Hypoxia-inducible factors regulate tumorigenic capacity of glioma stem cells. Cancer Cell, 2009. 15(6): p. 501-13.
21. Bourguignon, L. Y., et al., CD44 interaction with c-Src kinase promotes cortactin-mediated cytoskeleton function and hyaluronic acid-dependent ovarian tumor cell migration. J Biol Chem, 2001. 276(10): p. 7327-36.
22. Murakami, D., et al., Presenilin-dependent gamma-secretase activity mediates the intramembranous cleavage of CD44. Oncogene, 2003. 22(10): p. 1511-6.
23. Nagano, O., et al., Cell-matrix interaction via CD44 is independently regulated by different metalloproteinases activated in response to extracellular Ca(2+) influx and PKC activation. J Cell Biol, 2004. 165(6): p. 893-902.
24. Okamoto, I., et al., Proteolytic release of CD44 intracellular domain and its role in the CD44 signaling pathway. J Cell Biol, 2001. 155(5): p. 755-62.
25. Johansson, E., et al., CD44 Interacts with HIF-2a to Modulate the Hypoxic Phenotype of Perinecrotic and Perivascular Glioma Cells. Cell Rep, 2017. 20(7): p. 1641-1653.
26. Yuan, S. et al. Cryo-EM structure of a herpesvirus capsid at 3.1 Å. Science 360, eaao7283 (2018).
27. Manservigi, et al. HSV recombinant vectors for gene therapy. Open Virol. J. 4, 123-156 (2010).
28. Agelidis, et al. Cell entry mechanisms of HSV: what we have learned in recent years. Future Virol. 10, 1145-1154 (2015).
29. Weed, D. J. & Nicola, A. V. Herpes simplex virus membrane fusion. Adv. Anat. Embryol. Cell Biol. 223, 29-47 (2017).
30. Maruzuru, Y. et al. Role of herpes simplex virus 1 immediate early protein ICP22 in viral nuclear egress. J. Virol. 88, 7445-7454 (2014).
31. Shen, Y. & Nemunaitis, J. Herpes simplex virus 1 (HSV-1) for cancer treatment. Cancer Gene Ther. 13, 975-992 (2006).
32. Hartkopf, et al. Oncolytic virotherapy of gynecologic malignancies. Gynecol. Oncol. 120, 302-310 (2011).
33. Mozzi, A. et al. Simplexviruses successfully adapt to their host by fine-tuning immune responses. Mol. Biol. Evol. 39, msac 142 (2022).
34. Johnson, et al. Talimogene laherparepvec (T-VEC) for the treatment of advanced melanoma. Immunotherapy 7, 611-619 (2015).
35. Markert, et al. A phase 1 trial of oncolytic HSV-1, G207, given in combination with radiation for recurrent GBM demonstrates safety and radiographic responses. Mol. Ther. 22, 1048-1055 (2014).
36. Todo, T. et al. Intratumoral oncolytic herpes virus G47A for residual or recurrent glioblastoma: a phase 2 trial. Nat. Med. 28, 1630-1639 (2022).
37. Zhang, B. et al. Intratumoral OH2, an oncolytic herpes simplex virus 2, in patients with advanced solid tumors: a multicenter, phase I/II clinical trial. J. Immunother. Cancer 9, e002224 (2021).
38. Upasana, et al., TMIC-39. Targeting Extracellular Matrix Hyaluronic Acid-Cd44 Signaling Reduces Tumor Stemness And Sensitizes Tumor To Virotherapy And Enhances Therapeutic Potential For Cancer Treatment, Neuro-Oncology, Volume 24, Issue Supplement_7, November 2022, Page vii280, doi.org/10.1093/neuonc/noac209.1083.
39. Yoshihara, et al., Inferring tumour purity and stromal and immune cell admixture from expression data. Nat Commun, PMID: 24113773, (2013).
40. Jia, et al., Mining TCGA database for genes of prognostic value in glioblastoma microenvironment, Aging (Albany NY).10(4):592-605, PMID: 29676997 (2018).
41. Wen & Kesari, Malignant gliomas in adults, N Engl J Med., 359(8):877, PMID: 18669428 (2008).
42. Kasper, et al., Temporal characterization of hyaluronidases after peripheral nerve injury, PLOS ONE 18(8), PMID25999946 (2023).
43. Chen, et al., Identification of modules and functional analysis in CRC subtypes by integrated bioinformatics analysis, PLOS ONE, 14(8), PMID29747682 (2019).
44. Misra, et al., Interactions between Hyaluronan and Its Receptors (CD44, RHAMM) Regulate the Activities of Inflammation and Cancer. Front Immunol, 6:201, PMID: 25999946 (2015).
45. Terada, et al., Development of a rapid method to generate multiple oncolytic HSV vectors and their in vivo evaluation using syngeneic mouse tumor models, Gene Ther., (8):705-14, PMID: 16421599 (2006).
46. Agarwalla &Aghi, Oncolytic herpes simplex virus engineering and preparation, Methods Mol Biol., 797:1-19, PMID: 21948465 (2016).
47. Russell, et al., PTEN expression by an oncolytic herpesvirus directs T-cell mediated tumor clearance, Nat Commun., 9(1):5006, PMID: 30479334 (2018).
48. Otani, et al., Oncolytic HSV-Infected Glioma Cells Activate NOTCH in Adjacent Tumor Cells Sensitizing Tumors to Gamma Secretase Inhibition. Clin Cancer Res., 26(10):2381-2392, PMID: 32139403 (2020).
49. Agro & O'Brien, In vitro and in vivo Limiting Dilution Assay for Colorectal Cancer, Bio Protoc., 5(22):1-11, PMID: 29468185 (2015).

We claim:

1. An oncolytic virus engineered to express a nucleic acid sequence encoding a soluble extracellular portion of CD44.

2. The oncolytic virus of claim 1, wherein the soluble extracellular portion of CD44 comprises a truncated extracellular CD44 (sCD44) that can function as a dominant negative receptor to block signaling mediated by standard CD44 receptor.

3. The oncolytic virus of claim 1, wherein the virus is a herpes simplex virus.

4. The oncolytic virus of claim 1, wherein the virus is an oncolytic HSV-1.

5. The oncolytic virus of claim 1, wherein the administration of the oncolytic virus to subject with a tumor increases sensitivity of cells of the tumor to a DNA damaging agent.

6. The oncolytic virus of claim 5, wherein the DNA damaging agent is radiation.

7. The oncolytic virus of claim 5, wherein the DNA damaging agent is a chemotherapeutic agent.

8. A pharmaceutical composition comprising an effective amount of the oncolytic virus of claim 1.

9. A method of treating a subject with cancer comprising administering an effective amount of oncolytic virus of claim 1 to the subject.

10. The method of claim 9, further comprising treating the subject an second therapy.

11. The method of claim 10, wherein the second therapy comprises administering the subject a first DNA damaging agent.

12. The method of claim 11, wherein the DNA damaging agent is DNA-damaging chemotherapeutic agent.

13. The method of claim 11, wherein the DNA damaging agent is radiation.

14. The method of claim 11, wherein the second therapy comprises administering the subject a HIF-1α inhibitor.

15. The method of claim 9, wherein the administration of the oncolytic virus increases tumor cell sensitivity to tumor cell killing.

16. The method of claim 9, wherein the administration of the oncolytic virus increases immune cell infiltration of the tumor.

17. The method of claim 9, wherein the administration of the oncolytic virus decreases the number of stem cell properties of the cells within in a tumor.

18. The method of claim 9, wherein the administration of the oncolytic virus induces DNA damage in a tumor.

19. The method of claim 9, wherein the administration of the oncolytic virus downregulates MAPK signaling and inhibits the growth of the tumor.

20. The method of claim 9, wherein the cancer comprises a tumor, and the oncolytic virus is administered by intratumoral injection.
